(19) United States
(12) Patent Application Publication
(10) Pub. No.: US 2025/0322901 A1
(43) Pub. Date: Oct. 16, 2025

(54) SYSTEMS AND METHODS FOR METABOLITE IMPUTATION

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Eduard Reznik, New York, NY (US); Wesley Tansey, New York, NY (US); Sophie Jaro, New York, NY (US); Benjamin Freeman, New York, NY (US)

(21) Appl. No.: 18/868,601

(22) PCT Filed: May 25, 2023
(86) PCT No.: PCT/US2023/023584

§ 371 Date: Nov. 22, 2024

Related U.S. Application Data
(60) Provisional application No. 63/346,544, filed on May 27, 2022.

(51) Int. Cl. G16B 5/00

(52) U.S. Cl. CPC G16B 5/00

(57) ABSTRACT

Presented herein are systems and methods relating to imputing metabolite information. A method includes receiving a first and second metabolite dataset, normalizing, the first dataset and the second dataset, transforming, the normalized first and second datasets, and aggregating the normalized first dataset and the normalized second dataset to generate a first metabolite matrix, the first metabolite matrix missing a first relative abundance value. The method includes decomposing the first metabolite matrix into a second metabolite matrix and a third metabolite matrix to factorize the first metabolite matrix and generating a fourth metabolite matrix that is the product of the second metabolite matrix and the third metabolite matrix, wherein the fourth metabolite matrix including an imputed first relative abundance value.

### CROSS REFERENCE TO RELATED APPLICATION

This application claims the benefit of and priority to U.S. Provisional Patent Application 63/346,544, entitled “SYSTEMS AND METHODS FOR METABOLITE IMPUTATION,” filed May 27, 2022, the entirety of which is incorporated by reference herein.

### BACKGROUND

A computing device may employ computer vision techniques to impute at least one missing value from at least one dataset. In imputing the missing values, the computing device can transform data from the dataset.

### SUMMARY

At least one aspect of the present disclosure is directed to a method. The method can include receiving a first and second metabolite dataset, normalizing, the first dataset and the second dataset, transforming, the normalized first and second datasets, and aggregating the normalized first dataset and the normalized second dataset to generate a first metabolite matrix, the first metabolite matrix missing a first relative abundance value. The method includes decomposing the first metabolite matrix into a second metabolite matrix and a third metabolite matrix to factorize the first metabolite matrix and generating a fourth metabolite matrix that is the product of the second metabolite matrix and the third metabolite matrix, wherein the fourth metabolite matrix including an imputed first relative abundance value.

In some implementations, the method can include transforming, by the computing system, the fourth metabolite matrix to uniformly map the metabolite features of the fourth metabolite matrix between 0 and 1.

In some implementations, the first dataset is received from a first remote database and the second dataset is received from a second remote database.

In some implementations, the missing relative abundance value comprises the relative abundance value of a metabolite that was not measured in the first dataset or the second dataset.

In some implementations, the method can include applying, by the computing system, a loss function to identify a factorization value, the factorization value dictating a dimension of at least one of the second matrix or the third matrix.

In some implementations, the loss function can be a least squares error loss function, a hinge loss function, or a log loss function.

In some implementations, the method can include identifying, by the computer system, a third dataset likely to improve an accuracy of the imputed relative abundance value when normalized, transformed, and aggregated with the first dataset and the second dataset to generate an updated first metabolite matrix.

At least one aspect of the present disclosure is directed to a system. The system can include a computer system. The computer system can include a processing circuit having one or more processors and one or more memory, the memory storing instructions that, when executed by any one or more of the one or more processors, causes the one or more processors to receive, via a network from a remote database, a first dataset and a second dataset, the first dataset comprising data associated with a first set of metabolites, the second dataset comprising data associated with a second set of metabolites. The instructions can further cause the processor to normalize the first dataset and the second dataset via a total ion count (TIC) normalization and transform the normalized first dataset and second dataset, where the transformation ranking at least one left-censored entry of the first dataset or the second dataset. The instructions can further cause the processor to aggregate the normalized first dataset and the normalized second dataset to generate a first metabolite matrix, where the first metabolite matrix can be missing a first relative abundance value. The instructions can further cause the processor to decompose the first metabolite matrix into a second metabolite matrix and a third metabolite matrix to factorize the first metabolite matrix. The instructions can further cause the processor to generate a fourth metabolite matrix, where the fourth metabolite matrix is the product of the second metabolite matrix and the third metabolite matrix. The fourth metabolite matrix can include an imputed first relative abundance value.

In some implementations, the computer system can include the instructions further causing the processor to transform the fourth metabolite matrix to uniformly map the metabolite features of the fourth metabolite matrix between 0 and 1.

In some implementations, the first dataset can be received from a first remote database and the second dataset is received from a second remote database.

In some implementations, the missing relative abundance value can include the relative abundance value of a metabolite that was not measured in either the first dataset or the second dataset.

In some implementations, the computer system includes the instructions further causing the processor to apply a loss function to identify a factorization value, where the factorization value can dictate a dimension of at least one of the second matrix or the third matrix.

In some implementations, the loss function can be a least squares error loss function, a hinge loss function, or a log loss function.

In some implementations, the computer system can include the instructions further causing the processor to identify a third dataset likely to improve an accuracy of the imputed relative abundance value when normalized, transformed, and aggregated with the first dataset and the second dataset to generate an updated first metabolite matrix.

At least one aspect of the present disclosure is directed to non-transitory computer-readable medium storing instructions that, when executed by one or more processors cause the one or more processors to perform operations. The operations can include receiving a first and second metabolite dataset, normalizing, the first dataset and the second dataset, transforming, the normalized first and second datasets, and aggregating the normalized first dataset and the normalized second dataset to generate a first metabolite matrix, the first metabolite matrix missing a first relative abundance value. The method includes decomposing the first metabolite matrix into a second metabolite matrix and a third metabolite matrix to factorize the first metabolite matrix and generating a fourth metabolite matrix that is the product of the second metabolite matrix and the third metabolite matrix, wherein the fourth metabolite matrix including an imputed first relative abundance value.

In some implementations, the operations can include transforming the fourth metabolite matrix to uniformly map the metabolite features of the fourth metabolite matrix between 0 and 1.

In some implementations, the first dataset is received from a first remote database and the second dataset is received from a second remote database.

In some implementations, the missing relative abundance value comprises the relative abundance value of a metabolite that was not measured in the first dataset or the second dataset.

In some implementations, the operations can include applying a loss function to identify a factorization value, the factorization value dictating a dimension of at least one of the second matrix or the third matrix.

In some implementations, the operations can include identifying a third dataset likely to improve an accuracy of the imputed relative abundance value when normalized, transformed, and aggregated with the first dataset and the second dataset to generate an updated first metabolite matrix.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 depicts a flow diagram of a method for imputing metabolite data, according to an embodiment.

FIG. 2 depicts a block diagram of a computing system, according to an embodiment.

FIG. 3 depicts a block diagram of a normalization circuit of a computing system, according to an embodiment.

FIG. 4 depicts a block diagram of a rank transformation circuit of a computer system, according to an embodiment.

FIG. 5 depicts a block diagram of a non-negative matrix factorization circuit of a computer system, according to an embodiment.

FIG. 6 depicts a block diagram of a server system and a client computer system in accordance with an illustrative embodiment.

FIG. 7A depicts a flow diagram for imputing metabolite information, according to an embodiment.

FIG. 7B depicts a flow diagram for imputing metabolite information, according to an embodiment.

FIG. 7C depicts an example chart of aggregate data of metabolite imputation, according to an embodiment.

FIG. 8A depicts sample datasets generated by a metabolite imputation system, according to an embodiment.

FIG. 8B depicts an example chart of metabolite imputation performance, according to an embodiment.

FIG. 8C depicts an example chart of metabolite imputation performance, according to an embodiment.

FIG. 8D depicts an example chart of metabolite imputation performance, according to an embodiment.

FIG. 8E depicts an example chart of metabolite imputation performance, according to an embodiment.

FIG. 9A depicts an example chart of metabolites distinguishing tumor samples from normal samples, according to an example embodiment.

FIG. 9B depicts an example chart of metabolites distinguishing tumor samples from normal samples, according to an example embodiment.

FIG. 9C depicts an example chart illustrating the imputation of unmeasured features, according to an example embodiment.

FIG. 9D depicts an example chart of by-metabolite values with those features masked from a target dataset, according to an example embodiment.

FIG. 9E depicts an example chart of metabolite performance, in accordance with an example embodiment.

FIG. 9F depicts an example chart showing a relationship between actual and predicted metabolite ranks, according to an example embodiment.

FIG. 10A depicts example UMAP plots of a sample embedding matrix, in accordance with an example embodiment.

FIG. 10B depicts an example chart showing separation between tumor and normal samples, in accordance with an example embodiment.

FIG. 10C depicts an example chart showing feature embeddings of peptides and lipids from metabolites, in accordance with an example embodiment.

FIG. 10D depicts an example chart showing pathways enriched in certain embedding dimensions, in accordance with an example embodiment.

FIG. 11A depicts an example chart assessing imputation performance, in accordance with an example embodiment.

FIG. 11B depicts an example chart summarizing imputation performance by ionization mode, in accordance with an embodiment.

FIG. 11C depicts an example chart of metabolites predicted across ionization modes, in accordance with an embodiment.

### DETAILED DESCRIPTION

Following below are more detailed descriptions of various concepts related to, and embodiments of, systems and methods for data imputation, namely metabolite data. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.
Section A describes systems and methods for imputing missing metabolite data.
Section B describes systems and methods for metabolite imputation via rank-transformation and harmonization (“MIRTH”).
Section C describes a network environment and computing environment which may be useful for practicing various embodiments described herein.

### A. Systems and Methods for Imputing Missing Metabolite Data

Many metabolomics experiments measure a small fraction of metabolites in a given sample. Furthermore, many metabolomics experiments measure particular metabolites with little to no overlap between metabolites measured in other experiments. For example, many metabolomics experiments are conducted using mass spectrometry to measure a number of ions associated with a unique metabolite in a particular biological specimen, where accurate measurement requires particularized calibration of the study or mass spectrometry device to measure with maximum or desirable sensitivity with respect to the targeted metabolite or other metabolite of a similar chemistry. Consequently, the metabolite-specific nature of metabolomics experiments yields measured results focused narrowly on a relatively small number of metabolites while little to know actionable information related to other metabolites is learned by virtue of experimental design. Accordingly, there exists a desire to understand information (e.g., a relative abundance value) for other metabolites of the total metabolites present in a sample that may be latent in an experiment, but not measured or discernably understood because of experimental design. More specifically, there exists a need to impute latent information (e.g., a relative abundance value) for various metabolites that exists within a specimen that are not the focus of one experiment using information, latent or otherwise, from another experiment, for example.

An accurate and comprehensive understanding of metabolite information is crucial to understanding a metabolic pathway or metabolite biomarker that may be associated with disease, illness, therapeutic response, or some other biological phenomenon. With metabolomics experiments yielding information narrowly focused on a subset of metabolites, as-measured information from metabolomics experiments often cannot be leveraged for cross-dataset comparisons or similar studies to gain a broad or comprehensive understanding of metabolite information. Accordingly, medical understanding and medical diagnoses are limited by current methods in the field of metabolomics.

Systems and methods for imputing missing metabolite information are described herein. For example, systems and methods related to Metabolite Imputation via Rank-Transformation and Harmonization (MIRTH) can be used to impute partially-measured or entirely-unmeasured metabolite features across at least one metabolomics dataset. MIRTH can include a relative abundance factorization model that can be performed by one or more computer systems to impute metabolite features or learn relationships between various metabolite features using information from one or more metabolomics datasets. For example, MIRTH can transform relative abundance levels to normalized ranks such that the relative abundance levels in a metabolomics study can be mapped to a comparable scale to relative abundance levels of metabolites in separate metabolomics studies. Furthermore, MIRTH can implement rank transformation to identify covariation patterns between metabolites included in specimens of multiple studies (e.g., unmeasured metabolites for which latent information may be available) without making assumptions. MIRTH can apply a non-negative matrix factorization technique to the rank-transformed metabolomics data to factorize said data into one or more (e.g., two) low-dimensional matrices that describe the latent structure between samples and metabolite features. The latent structure between samples and metabolite features described in these matrices can reveal a correlative relationship between one or more metabolites across multiple metabolomics datasets. By imputing missing metabolite measurements, MIRTH can recover rank-normalized metabolite abundances which are biologically significant or of clinical importance without requiring additional experiments to specifically target additional metabolites. For example, MIRTH can facilitate the generation of hypotheses and conclusions regarding metabolite abundance levels or interrelationships between metabolites by imputing missing information from previously-conducted studies. Furthermore, by imputing missing metabolite measurements from one or more datasets, MIRTH provides a more complete understanding of the metabolic nature of a particular sample. For example, MIRTH can be used to understand how a metabolome of one type of sample (e.g., tumorous sample) and another type of sample (e.g., normal sample) vary or are similar across multiple sample types (e.g., various cancer types).

MIRTH can impute missing metabolite measurements within a single metabolomics dataset or within multiple metabolomics datasets. For example, MIRTH can impute missing metabolite measurements within a single dataset, where the single dataset includes measurements associated with at least one metabolite and latent information regarding a plurality of unmeasured metabolites. In this scenario, MIRTH can reveal information regarding the plurality of unmeasured metabolites by normalizing, transforming, and factorizing the dataset. For example, by normalizing, transforming, and factorizing the dataset, MIRTH can impute metabolite information (e.g., a relative abundance value) for one or more of the plurality of unmeasured metabolites such as amino acids, carbohydrates, cofactors, vitamins, energy carriers, lipids, nucleotides, peptides, xenobiotics, or other metabolites, for example. MIRTH can impute metabolite measurements of entirely unmeasured metabolites in a manner that preserves a relationships between biologically significant metabolites when data is imputed across more than one dataset.

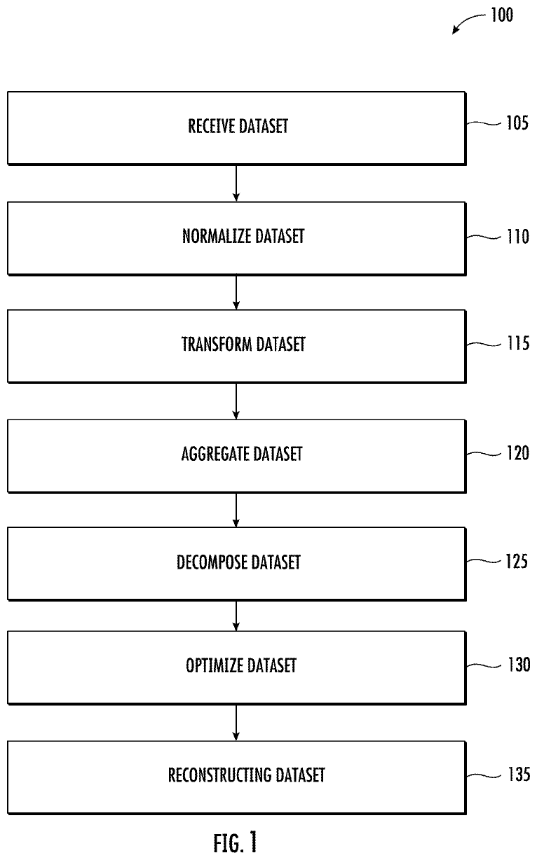

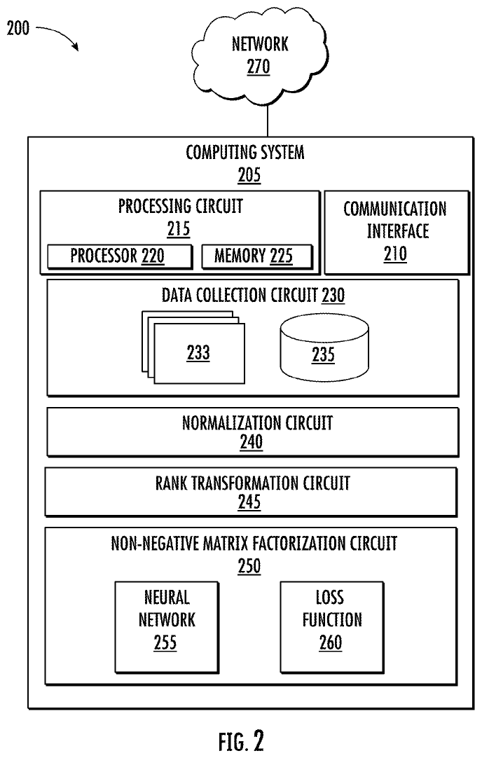

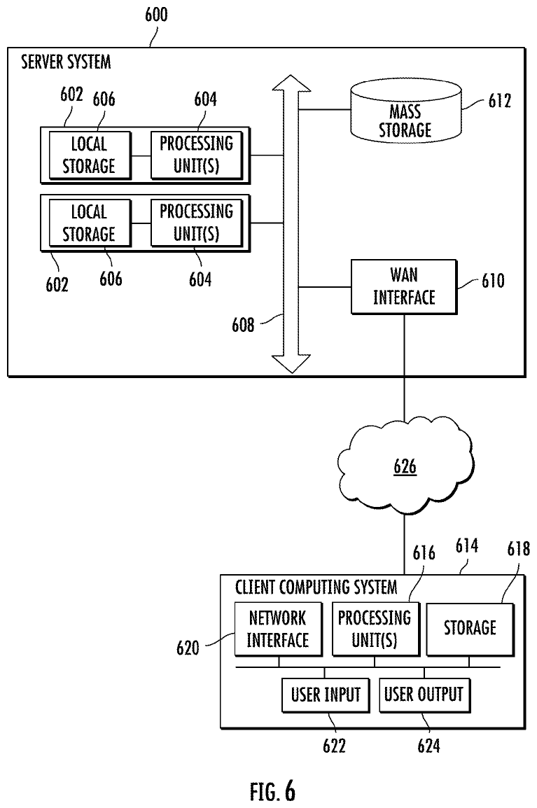

Referring now to FIG. 1, a method for imputing missing metabolites is shown. The method 100 method 100 can include one or more of processes 105-130 and can be performed by a computing system, such as the system 200 shown in FIG. 2, among others, or the server system 600 of FIG. 6. The system 200 can include a computing system 205 coupled with a network 270. The computing system 205 can include a communication interface 210, a processing circuit 215, a data collection circuit 230, at least one database 235, a normalization circuit 240, a rank transformation circuit, and a non-negative matrix factorization circuit 250. The processing circuit 215 can include a processor 220 and a memory 225. In other embodiments, the computing system 205 may include any number of processors and/or memory such that the functionality and processes of the computing system 205 may be optionally distributed across multiple processors or devices. The data collection circuit 230 can include the database 235 or one or more additional databases 235.

In various examples, the data collection circuit 230 can collect data from at least one metabolomics datasets, where the one or more metabolomics datasets are identified as requiring imputation, possessing latent information of interest in a metabolite imputation operation, or for some other reason. The data collection circuit 230 can store the received metabolomics datasets in the database 235. The normalization circuit 240 can process at least some of the data received by the data collection circuit 230. The rank transformation circuit 245 can perform a rank transformation operation to rank-transform data associated with at least one dataset. For example, the rank transformation circuit 245 can rank-transform normalized data received from the normalization circuit 240. The non-negative matrix factorization circuit 250 can aggregate rank-transformed datasets to create a matrix. The non-negative matrix factorization circuit 250 can decompose the matrix to create a second matrix and a third matrix. The non-negative matrix factorization circuit 250 can apply a loss-function to data, such as a least squares error function. The non-negative matrix factorization circuit 250 can reconstruct a fourth matrix using the third matrix and the fourth matrix. Using the fourth matrix, the metabolite imputation system 200 and the computing system 205 can generate hypotheses or conclusions about previously unmeasured metabolites, for example.

The computing system 205 may be used by a user, such as a scientist, researcher, or medical professional. In one example, the computing system 205 is structured to exchange data over the network 270 via the communication interface 210, execute software applications, access websites, etc. The computing system 205 can be a personal computing device or a desktop computer, according to one example. The computing system 205 can be a cloud-computing system, a mobile device, or some other computing device.

The communication interface 210 can include one or more antennas or transceivers and associated communications hardware and logic (e.g., computer code, instructions, etc.). The communication interface 210 is structured to allow the computing system 205 to access and couple/connect to the network 270 to, in turn, exchange information with another device (e.g., a remote database 235, a remotely-located computing system, a cloud computing system, etc.). The communication interface 210 allows the computing system 205 to transmit and receive internet data and telecommunication data another device, for example. Accordingly, the communication interface 210 includes any one or more of a cellular transceiver (e.g., CDMA, GSM, LTE, etc.), a wireless network transceiver (e.g., 802.11X, ZigBee®, WI-FI®, Internet, etc.), and a combination thereof (e.g., both a cellular transceiver). Thus, the communication interface 210 enables connectivity to WAN as well as LAN (e.g., Bluetooth®, NFC, etc. transceivers). Further, in some embodiments, the communication interface 210 includes cryptography capabilities to establish a secure or relatively secure communication session between other systems such as a remotely-located computer system, a second mobile device associated with the user or a second user, the a patient's computing device, and/or any third-party computing system. In this regard, information (e.g., confidential patient information, images of tissue, results from tissue analyses, etc.) may be encrypted and transmitted to prevent or substantially prevent a threat of hacking or other security breach.

The processing circuit 215 can include the processor 220 and the memory 225. The processing circuit 215 can be communicably coupled with the data collection circuit 230, the normalization circuit 240, the rank transformation circuit 245, the non-negative matrix factorization circuit 250, or the metabolite application circuit 265. For example, the processing circuit 215 can include one or more of the data collection circuit 230, the normalization circuit 240, the rank transformation circuit 245, the non-negative matrix factorization circuit 250, or the metabolite application circuit 265. The data collection circuit 230, the normalization circuit 240, the rank transformation circuit 245, the non-negative matrix factorization circuit 250, or the metabolite application circuit 265 can be located within or remotely from computing system 205. The data collection circuit 230, the normalization circuit 240, the rank transformation circuit 245, the non-negative matrix factorization circuit 250, or the metabolite application circuit 265 can be executed or operated by the processor 220 of the processing circuit 215. The processor 220 can be coupled with the memory 225. The processor 220 can be a general purpose or specific purpose processor, an application specific integrated circuit (ASIC), one or more field programmable gate arrays (FPGAs), a group of processing components, or other suitable processing components. The processor 220 is configured to execute computer code or instructions stored in the memory 225 or received from other computer readable media (e.g., CDROM, network storage, a remote server, etc.).

The memory 225 can include one or more devices (e.g., memory units, memory devices, storage devices, etc.) for storing data and/or computer code for completing and/or facilitating the various processes described in the present disclosure. The memory 225 may include random access memory (RAM), read-only memory (ROM), hard drive storage, temporary storage, non-volatile memory, flash memory, optical memory, or any other suitable memory for storing software objects and/or computer instructions. The memory 225 may include database components, object code components, script components, or any other type of information structure for supporting the various activities and information structures described in the present disclosure. The memory 225 may be communicably connected to the processor 220 via processing circuit 215 and may include computer code for executing (e.g., by the processor 220) one or more of the processes described herein. For example, the memory can include or be communicably coupled with the processor 220 to execute instructions related to the data collection circuit 230, the normalization circuit 240, the rank transformation circuit 245, the non-negative matrix factorization circuit 250, or the metabolite application circuit 265. In one example, the memory 225 can include or be communicably coupled with the data collection circuit 230, the normalization circuit 240, the rank transformation circuit 245, the non-negative matrix factorization circuit 250, or the metabolite application circuit 265. The data collection circuit 230, the normalization circuit 240, the rank transformation circuit 245, the non-negative matrix factorization circuit 250, or the metabolite application circuit 265 can be stored on a separate memory device located remotely from the computing system 205 that is accessible by the processing circuit 215 via the neural network 255.

At process 105, the method 100 can receive at least one dataset. For example, the method can be performed by the computing system 205 that receives one or more datasets 233. Each of the datasets can be a metabolomics dataset containing data associated with one or more metabolites of a group of metabolites present within at least one sample. For example, each of the datasets 233 can include data associated with a relatively small number (e.g., 0.5-5%, 10%, 15%, etc.) of metabolites present within a single biological sample or within multiple biological samples. The datasets 233 can be received by the data collection circuit via the memory 225 of the computing system, another memory device (e.g., a database of the computing system 205), another computing system (e.g., a server system associated with a medical center or hospital storing encrypted patient data), a remote database, or some other location. The data collection circuit 230 can receive the one or more datasets 233 via the communication interface 210 of the computing system.

A biological sample can include at least one missing metabolite, where a missing metabolite can be a metabolite that exists within the sample in a measurable quantity, but that which are outside of the scope of a metabolomics experiment and thus have neither been measured nor included in the dataset 233. The datasets 233 can include one or more left-censored metabolites. The left-censored metabolites can be or include metabolites that may be measured in one sample but not in another sample of the dataset 233, perhaps because the relative abundance of that metabolite is below a threshold as to not be measured or measurable. Accordingly, the datasets 233 can include metabolomics data excluding certain missing metabolites and excluding certain left-censored data.

The data collection circuit 230 can automatically receive data. For example, the data collection circuit can receive at least one dataset parameter. The dataset parameter indicating some characteristic of the dataset 233, such as the presence of a particular measured metabolite within the dataset, the presence of a group of measured metabolites within the dataset 233, the size of the dataset 233 (e.g., the approximate number of measured and unmeasured metabolites within a dataset), or some other parameter. A user can provide the parameter to the computing system 205 via the communication interface 210 (e.g., a keyboard, a graphical user interface, etc.). The data collection circuit 230 can analyze imputed metabolite data (e.g., a dataset generated by the non-negative matrix factorization circuit 250) to identify a dataset parameter, such as the presence of a particular imputed metabolite within a dataset, the accuracy of the imputed metabolite data based on a known dataset, or some other characteristic. Based on the dataset parameter, the data collection circuit 230 can poll or search one or more databases 235, remotely located computing systems or databases, etc. in order to identify datasets 233 that meet a certain criteria according to the dataset parameter. For example, the data collection circuit 230 can automatically collect datasets 233 from one or more locations that include (or exclude) certain data or meet a certain criteria as prescribed by the dataset parameter.

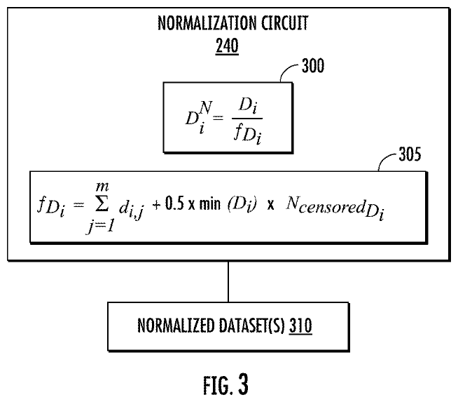

The method 100 can include normalizing a dataset at process 110. For example, the computing system 205 can perform the process 110 to normalize data associated with one or more datasets 233. The normalization circuit 240 of the computing system 205 can receive data from the data collection circuit 230. The normalization circuit 240 can manipulate, transform, edit, or modify the data of the received one or more datasets 233. As depicted in FIG. 3, among others, the normalization circuit 240 can normalize the data within the dataset according to a normalization function 300. The first function 300 can be configured to control for variation in sample loading in the received datasets 233. For example, the normalization circuit 240 can control for variation in the received datasets 233 by normalizing an ion count for every metabolite entry present in a sample according to the normalization function 300 for at least one received metabolomics dataset. The normalization circuit 240 can generate a total ion count (TIC) sample vector by dividing an unnormalized sample vector by a TIC normalizer value f_{Di}. The TIC normalizer value can be determined according to the TIC normalizer value function 305, for example. The TIC normalizer value function 305 can consider the total number N_{censored} of left-censored entries in a sample, where left-censored entries are those metabolite entries that are missing in certain datasets 233 but are measured in other datasets 233. For example, a left-censored metabolite entry represents the presence of a metabolite in a sample, but at a relative abundance level that is below a threshold value and is thus not measured within the dataset 233. The normalization circuit 240 can thus normalize at least one dataset 233 via normalization function 300 while taking into account the presence of left-censored values via the TIC normalizer value f_{Di}. The normalization circuit 240 can generate at least one normalized dataset 310, where the normalized dataset 310 can be the dataset 233 that has been normalized according to the normalization function 300, for example.

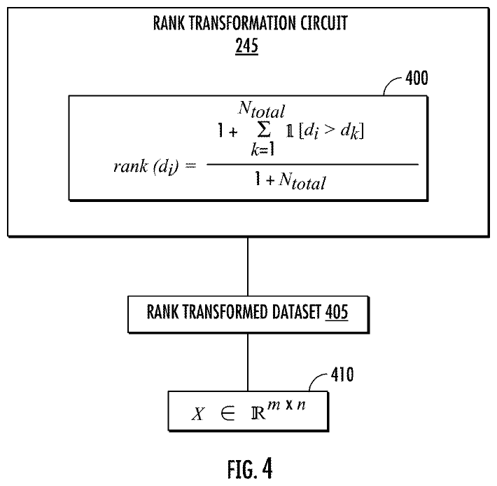

The method 100 can include transforming at least one dataset at process 115. For example, the computing system 205 can perform the process 115. The rank transformation circuit 245 of the computing system 205 can perform the process 115. For example, the rank transformation circuit 245 can receive at least one normalized dataset 310 from the normalization circuit 240. The rank transformation circuit 245 can rank the relative abundance of metabolites within the normalized dataset 310 according to a rank transformation function 400, as shown in FIG. 4 among others. For example, the rank transformation circuit 245 can rank the relative abundance value of the metabolites in multiple normalized datasets 310 to generate rank transformed dataset 405 that distributes the relative abundance values for each metabolite in the dataset in a similar way. By transforming the normalized datasets 310 to rank the data by relative metabolite abundance values via the rank transformation circuit 245, the relative abundance values of various metabolites can be compared within the same dataset or between multiple datasets. For example, the samples with a high ion count (e.g., a high relative abundance value for a given metabolite) are ranked highest in the rank-transformed data, while samples having a low ion count (e.g., a low relative abundance value for a given metabolite) are ranked low.

Left-censored values within the normalized dataset 310 can be ranked last or can be tied for the last rank in the rank transformed dataset 405. In some examples, the left-censored values in a normalized dataset 310 or other dataset manipulated by the rank transformation circuit 245 can be rank transformed according to a second rank transformation function 410. The second rank transformation function 410 can rank the left-censored data halfway or approximately halfway between a minimum rank of an uncensored sample as ranked by the rank transformation function 405 and 0. The rank transformation circuit 245 can rank a normalized dataset 310 including only uncensored metabolites uniformly from 0 to 1, according to some examples. For example, the rank transformation circuit 245 can rank transform one or more normalized datasets 310 such that metabolites (e.g., metabolite features) in each dataset can have the same or a similar marginal distribution that can be conditioned on having the same sample size.

The method 100 can include aggregating at least one dataset at process 120. For example, the process 120 can include aggregating one or more rank transformed datasets 405 that are generated by the rank transformation circuit 245 to form the matrix 410. The matrix 410 can be an aggregation of the datasets 405 and can include rows containing samples from each of the rank transformed datasets 405. The matrix 410 can include columns containing features (e.g., metabolites) from each of the rank transformed datasets 405. For example, the matrix 410 can include columns corresponding to the complete set of metabolites measured across multiple experiments and samples, including where certain samples do not include a measurement for a particular metabolite or feature. Accordingly, the matrix 410 can be a sparse matrix (i.e., incomplete, having missing data) of relative abundance values of various metabolites. The sparse nature of the matrix 410 can be a reflection of the missing features (e.g., metabolites) that were excluded from metabolomics experiments associated with the datasets 233 received at process 105. Accordingly, the matrix 410 of relative abundance values may not be complete because the datasets 233 (and by implication the datasets 310 and 405) may not contain measurements or data for each of the metabolites (e.g., features) in the respective samples. The method 100 and the metabolite imputation system 200 seeks to impute (e.g., predict, estimate, determine) relative abundance values for these missing metabolites. Though the matrix 410 can be sparse, the matrix 410 can be a non-negative (e.g., having values between 0 and 1) and high-dimensional data matrix (e.g. 10 rows or columns, 100 rows or columns, 1000 rows or columns, etc.).

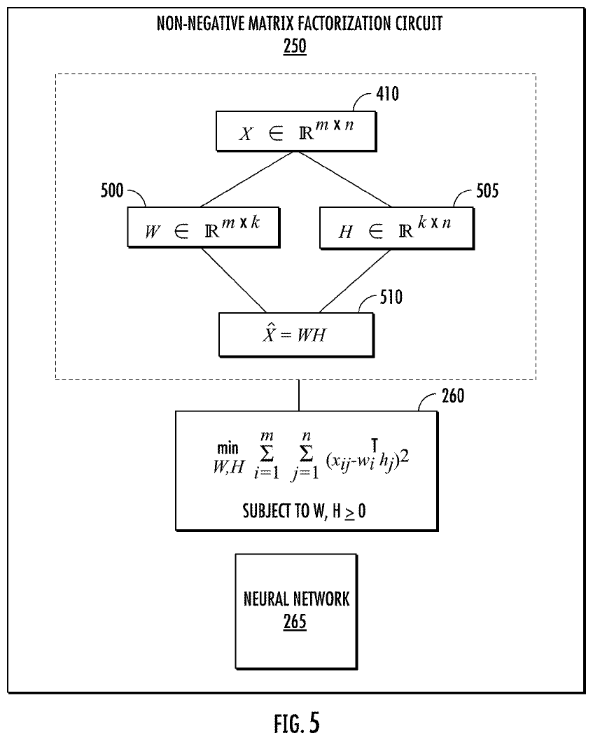

The method 100 can include decomposing a dataset at process 125. For example, the process 125 can be performed by the non-negative matrix factorization circuit 250 of the computing system 205 as shown in FIG. 5, among others. The non-negative matrix factorization circuit 250 can receive the matrix 410 from the rank transformation circuit after the matrix 410 is created by aggregating datasets 405 at process 120. The non-negative matrix factorization circuit 250 can be configured to obtain a low-rank approximation of non-negative, high-dimensional data matrices, such as the matrix 410. The non-negative matrix factorization circuit 250 can decompose the high-dimensional matrix 410 into a matrix 500 and a matrix 505. The matrix 500 and the matrix 505 can be low-dimensional matrices. For example, the non-negative matrix factorization circuit 250 can decompose the matrix 410 into the matrix 500 where rows of the matrix 505 contain samples (e.g., all or some portion of the samples of the matrix 410) but the columns include or describe a relative contributions among one or more embedding vector of each sample. The matrix 500 with columns describing embedding vectors can reveal clustering among samples, for example. The matrix 505 can include columns containing features (e.g., metabolites). For example, the matrix 505 can include all or some of the features of the matrix 410. The rows of the matrix 505 can include k embedding vectors for each feature and can describe the relative contribution of the features to an embedding vector.

The method 100 can include optimizing a dataset at process 130. For example, the non-negative matrix factorization circuit 250 of the computing system 205 can perform operations associated with process 130. To determine an optimal number of embedding dimensions k when performing the decomposition of the matrix 410, the non-negative matrix factorization circuit 250 can perform a v-fold cross-validation operation. In order to evaluate performance, it is necessary to identify known parameters that can be used to test the accuracy of a factorization for a given k value. Accordingly, the non-negative matrix factorization circuit 250 can receive the datasets 405 from the rank transformation circuit 245 or the normalized datasets 310 from the normalization circuit 240. Using the one or more datasets 310, 405, the non-negative matrix factorization circuit 250 can determine, one or more metabolites (e.g., 9, 13, 20) that are available for cross-wise validation, namely the metabolites in each dataset that are also measured in at least one other dataset. With the metabolites available for cross-wise validation known, the non-negative matrix factorization circuit 250 can decompose the matrix 410 into the matrix 500 having k columns and the matrix 505 having k rows, where k can be some value (e.g., a value between 1-60, a value between 1-80, or some other number). With the matrix 410 decomposed into matrices 500 and 505, the non-negative matrix factorization circuit 250 can use a loss function 260 to determine an error value associated with the factorization operation (i.e., the error associated with factorizing matrix 410 into matrices 500 and 505 for a particular k). The loss function 260 can be a least squares error loss function, a hinge loss function, a log loss function, or some other loss function. For example, the loss function 260 can be a least squares error loss function. In examples where the loss function 260 is an entry-wise sum of losses, the missing values (e.g., those values that are not available for cross-wise validation because they are not present in another dataset) can be omitted from the loss function 260.

The non-negative matrix factorization circuit 250 can optimize the factorization of the matrix 410 to improve the accuracy of imputed metabolite values. For example, the non-negative matrix factorization circuit 250 can optimize matrices 500 by performing factorizing the matrix 410 (e.g., decomposing the matrix 410) into a plurality of matrices 500 and 505 and computing an error value for multiple k values within a range of k values. The non-negative matrix factorization circuit 250 can determine a k value that is associated with the lowest error value. The non-negative matrix factorization circuit 250 can use the identified k value to decompose the matrix 410 into matrices 500 and 505, where the matrix 500 has rows corresponding to samples and k columns of embedding vectors and the matrix 505 has columns corresponding to metabolites (or features) and k rows of embedding vectors.

The method 100 can reconstruct a dataset at process 135. For example, the non-negative matrix factorization circuit 250 of the computing system 205 can perform the process 135. The non-negative matrix factorization circuit 250 can generate an imputed metabolite matrix 510. For example, the non-negative matrix factorization circuit 250 can generate the imputed metabolite matrix 510 by multiplying the matrix 500 with the matrix 505. The imputed metabolite matrix 510 can include imputed values for each of the missing metabolite values. While the matrix 410 can be a sparse matrix as described above, the matrix 510 may not be sparse such that each missing metabolite value has been imputed by virtue of the factorization operation performed by the non-negative matrix factorization circuit 250 to generate factorized matrices 500 and 505 that produce the imputed metabolite matrix 510.

The non-negative matrix factorization circuit 250 can include a neural network 255. For example, the non-negative matrix factorization circuit 250 can include a neural network 255 that can perform various functions of the non-negative matrix factorization circuit 250. For example, the neural network 255 can identify metabolites that are available for use in dataset-wise cross validation. In another example, the neural network 255 can predict an appropriate k value out of a range of k values based on a characteristic of the dataset 405 or a characteristic of one or more datasets 310 or 233. The neural network 255 can include a training dataset including data or information related to metabolites, previous metabolite imputation operations performed by the non-negative matrix factorization circuit 250, or otherwise. The neural network 255 can also be deeply pre-trained neural network. In some examples, the neural network 255 can be separate from the non-negative matrix factorization circuit 250. In such examples, the neural network 255 can be used to identify datasets 233 stored in a memory on the computing system 205, stored remotely, or otherwise associated with the metabolite imputation system 200 that may be suitable for a metabolite imputation operation such as the operations of method 100.

The computing system 205 or the metabolite imputation system 200 more generally can use the matrix 510 to generate graphics related to the significance or interrelationship of various metabolites based on imputed relative abundance values, make hypotheses about the relationship between a measured metabolite and an imputed relative abundance value, make medical recommendations based on an imputed relative abundance value, or otherwise leverage imputed information related to metabolites. For example, the computing system 205 can differentiate, based on the presence of an imputed metabolite in a sample, between a healthy sample and an unhealthy (e.g., cancerous). In particular, the computing system 205 can predict the presence of tumor-enriched metabolites or tumor-depleted metabolites in one dataset where those particular metabolites were previously unmeasured. In another example, the computing system 205 can use imputed relative abundance values to identify and display to a user (e.g., via a display device, graphical user interface, wireless transmission to a mobile device, etc.) information relating to a correlative relationship between metabolites across datasets.

### B. Systems and Methods for Metabolite Via Rank-Transformation and Harmonization (“Mirth”)

### 1. Background

Large-scale quantification of metabolite pool sizes (“metabolomics”) is a powerful approach for the mechanistic investigation of metabolic pathway activity and the identification of metabolic biomarkers of disease and therapeutic response [1-5]. By observing how metabolite levels are altered in various physiological conditions, metabolomics can reveal the role of metabolites in homeostasis, in disease, or in response to perturbations [6].

The bulk of large-scale metabolomics data in biology research is now generated using mass spectrometry [7]. This technology ultimately reports the number of measured ions associated with a unique metabolite in a given biological specimen. To accurately identify metabolites, targeted metabolomics studies must be calibrated for maximum sensitivity for specific classes of metabolites with similar chemical properties [8]. Consequently, each metabolomics platform can only measure a sub-set of the entire assortment of metabolites in a specimen. Metabolomics assays operated in different laboratories often measure sets of metabolites with little over-lap. For example, in a pan-cancer series of eleven metabolomics datasets [9], only 23 out of 935 metabolites were measured across all samples. This lack of overlap restricts cross-dataset comparisons and impedes the discovery of general principles of metabolite regulation across datasets. The goal of this work is to enable cross-dataset comparisons by developing a method to impute missing metabolites between datasets.

Imputing missing values is specifically challenging in metabolomic data analysis because metabolite levels are reported in arbitrary units, which we refer to as relative abundance. A relative abundance level only contains information about the concentration of a metabolite in a sample relative to all other measurements of that metabolite in that dataset. These levels are not comparable between different metabolites in the same dataset, nor are they comparable to the measurements of the same metabolite in different datasets. The lack of a shared measurement scale between metabolites and datasets prevents the application of existing imputation methods that assume a common basis (e.g. probabilistic PCA [10]). Others have developed methods for the imputation of single metabolomics datasets, including some based on k-nearest neighbor imputation [11, 12], quantile regression imputation of left-censored data & random forest imputation [13], kernel-weighted least squares imputation [14], and multivariate imputation by chained equations [12]. These methods impute left-censored values-missing values arising when a metabolite level falls below a detection threshold in a subset of samples-within a single dataset [13].

In contrast to the above-mentioned work, we consider here a related but larger and more challenging class of problems related to imputing entirely-unmeasured metabolite features across datasets. Here, we present Metabolite Imputation via Rank-Transformation and Harmonization (MIRTH), a relative abundance matrix factorization model that learns relationships between metabolite levels in one or more metabolomics datasets. MIRTH's key insight is that transforming relative abundance levels to normalized ranks maps every measurement to a comparable scale between metabolites and across batches. Critically, rank transformation enables MIRTH to discover patterns of covariation between metabolite pools that are shared across datasets without making assumptions about the relative concentrations of the same metabolite across datasets. MIRTH factorizes rank-transformed metabolomics data into two low-dimensional embedding matrices (FIG. 1). These embeddings describe the latent structure between samples and metabolite features. By compressing the information contained in the space of all metabolite features measured across all datasets into low-dimensional embeddings, MIRTH discovers correlative relationships among metabolites across datasets. These correlations enable the imputation of unmeasured features in each dataset. Similar matrix factorization techniques have previously been applied to a variety of data modalities [15], including gene expression data [16-18], protein sequences [18], and genomic data [19] for clustering analysis and class discovery.

We evaluate the performance of MIRTH in a pan-cancer series of nine metabolomics datasets. MIRTH achieves high accuracy in in silico experiments predicting ranks. In each of our nine batches of experimental data, a proportion of simulated-missing metabolites entirely masked from a batch are imputed well by MIRTH. In kidney cancer data with paired tumor and normal tissues, MIRTH correctly predicts un-measured tumor-enriched and tumor-depleted metabolites in one dataset by transferring information from a second dataset where those metabolites were measured. MIRTH also accurately imputes metabolites across ionization modes, enabling the imputation of unmeasured metabolites across chemically distinct classes. By in-creasing the available information about the metabolome, MIRTH increases the hypothesis-generating potential of existing datasets while revealing new information embedded in existing metabolomics data.

### 2. Results

We completed a series of benchmark studies to assess the performance of MIRTH in different imputation tasks. We evaluated the performance of MIRTH on a collection of nine previously-published mass-spectrometry metabolomics datasets [9]. Metabolite names were harmonized by maximizing consistency across multiple metabolite identifiers, as previously described [9]. Details on the number of samples and features in each dataset are reported in Table S1.

For each benchmark, we measured the concordance between the true and imputed ranks of metabolite samples in held-out data. Across experiments, we found MIRTH performed well in high sample-to-metabolite scenarios. In contrast, MIRTH performed poorly when there were insufficient samples to train on and when samples were highly censored. Furthermore, we found that a subset of metabolites were reproducibly well-imputed across different datasets and imputation tasks, ascribing a quantitative metric of confidence to MIRTH's predictions.

2.1 MIRTH Recovers Missing Metabolites within Metabolomics Datasets

We first verified that MIRTH accurately imputed missing measurements within a single dataset. This represented the most straightforward imputation task because there were no batch effects associated with merging of data from two or more distinct datasets. We performed an in silico experiment, simulating a scenario where a set of metabolites was not measured in a subset of samples in the dataset. First, we randomly select 50% of the samples to serve as hold-out samples. Next, we randomly selected 10% of all the metabolites to serve as hold-out metabolites (FIG. 2a). We masked the hold-out metabolites in the hold-out samples to simulate that they were not measured in half the dataset. This effectively split the dataset into two pseudo-datasets, where a smaller set of features was measured in the hold-out pseudo-dataset.

For each of the 9 benchmarking datasets (Table S1), we split the data as described above and applied MIRTH to impute the held-out values (FIG. 2a and Methods). Since the held-out values in the dataset were actually known, the performance of MIRTH was assessable by comparing the actual and predicted ranks in the simulated-missing features. We repeated this experiment 200 times for each of the 9 datasets, randomly selecting a set of metabolites in a random set of samples to mask in each iteration. The optimal number of embedding dimensions, chosen through cross-validation separately for each dataset, ranged between 3 and 37 (Figure S1a). We also evaluated the performance of MIRTH and its sensitivity to dataset size and feature missingness using simulated metabolomics data. The method and results of testing single-set performance on simulated data are available in the Supplementary Information section.

When 10% of features were simulated missing in each trial, MIRTH successfully predicted the abundance of approximately 89% of metabolites (significant positive correlation with the true ranks in at least 90% of trials, ρ>0, q<0.05, BH-corrected, FIG. 2b). This proportion ranged from 11% in BrCa2 (likely due to its small number of samples) to 100% in COAD (Table S2). Variation in MIRTH performance across datasets was partly explained by dataset size; imputation performance was better for larger datasets (FIG. 2c). The proportion of metabolites that were predicted with ρ>0.3 in each dataset likewise increased as the sample size of the dataset increased (FIG. 2d). This suggested that poor predictions may be the result of an insufficient quantity of data from which MIRTH could learn. Similarly, when the proportion of features simulated as missing was increased, imputation performance worsened (Figure S1b). This result was expected since higher proportions of masked metabolites leave less data from which MIRTH can learn. We also investigated factors associated with each metabolite feature which might influence imputation accuracy. We observed that metabolite features with larger coefficients of variation (in the raw, non-rank-transformed data) tended to have lower imputation accuracy (Figure S1c). Similarly, predictions of features with a greater number of censored measurements (i.e. those where more measurements were below the detection threshold) scored lower. This likely arose from a poor correlation be-tween censored values' tied-for-last ranks at the input and their uniformly-mapped ranks at the output (Figure S1d).

A total of 306 metabolites were reproducibly well-predicted by MIRTH across multiple datasets, meaning that they were measured in at least 4 datasets and well-predicted in at least three-quarters of the datasets in which they were measured (FIG. 2e). Among these well-predicted metabolites were 84 amino acids, 22 carbo-hydrates, 16 cofactors and vitamins, 5 energy carriers, 120 lipids, 28 nucleotides, 21 peptides, 8 xenobiotics, and 2 uncharacterized metabolites. Well-predicted metabolites were enriched in specific metabolite classes, including dipeptides, proteinogenic amino acids and various lipid subsets (Table S3). For example, palmitate and methionine, which were measured in 8 and 9 datasets respectively, were both well-predicted in 8 experiments (FIG. 2c). While these benchmarking experiments were conducted in a setting where the ground truth was known, the observation that certain metabolites were reproducibly well-imputed in different settings suggests that imputation of their abundance by MIRTH in settings where the true abundance is unknown should be associated with additional confidence relative to all other metabolites. Furthermore, the ability of MIRTH to recover ranks of sub-sets of samples of metabolites in a single dataset motivated the use of MIRTH to impute entirely missing metabolites in a single dataset by learning from a matrix of aggregate datasets.

### 2.2 MIRTH Recovers Missing Metabolites by Transferring Knowledge Across Datasets

To determine if imputation of entirely unmeasured features could produce biologically sound predictions of missing metabolites, we applied MIRTH to two independent kidney cancer datasets, RC12 and RC3, each consisting of both tumor and adjacent normal tissue samples. We reasoned that metabolites distinguishing tumor and normal tissues should be highly concordant across both datasets. To test this assumption, we calculated the differential abundance of all metabolites (tumor vs. normal) in RC12 and RC3 (Figure S2). Of the 169 metabolites measured in both RC12 and RC3 that showed statistically significant differences between tumor and normal in both datasets, we observed that 159 (94%) showed identical changes, including canonical metabolites such as glutathione (GSSG and GSH), lactate, NAD+, and fructose (q<0.05, Wilcoxon test, FIG. 3a). This demonstrated that the metabolite differences between tumor and normal samples in these datasets were comparable. Next, we repeated the analysis above considering metabolites measured in RC12, but entirely unmeasured in RC3, which we called test metabolites. After joint imputation of RC12 and RC3 with MIRTH, we compared the differential abundance of test metabolites in RC12 (where they were measured) and RC3 (where their true abundance is unmeasured, but where they have been imputed by MIRTH, Figure S2). Doing so, we again observed a strong correlation between differential abundance (tumor vs. normal) in test metabolites in RC12 and RC3. Out of 252 test metabolites, there were 235 (93%) significant and consistently differentially abundant metabolites, including glucose-1-phosphate (GIP), fructose-6-phosphate (F6P), fructose-1-phosphate (FIP), and gamma-aminobutyric acid (GABA). Only 17 metabolites inconsistently distinguished tumor and normal samples (q<0.05, Wilcoxon test, FIG. 3b). This analysis confirms that MIRTH preserves relationships between sample types and biologically important metabolites when imputing data across datasets, and suggests that MIRTH can be successfully applied to impute the ranks of entirely unmeasured metabolites in metabolomic data.

To further assess the ability of MIRTH to accurately impute missing features across datasets, we designated one of the nine datasets under analysis as the tar-get, from which we completely masked a set of features to simulate as unmeasured. MIRTH was then applied to impute these unmeasured features, using data from the remaining eight datasets (and therefore testing the performance of MIRTH in the presence of a dataset-specific batch effect). We conducted nine such experiments, where each dataset was the target for one experiment (FIG. 3c). We repeated each experiment for 200 trials for each target dataset, randomly selecting 10% of features to simulate as missing each time. The optimal number of embedding dimensions ranged between 26 and 48, but equivalent-to-optimal performance could be achieved with approximately 30 dimensions (Figure S3a). Performance was evaluated similarly to the within-dataset imputation, comparing imputed and true ranks of the features simulated as missing. We also evaluated MIRTH's across-set imputation performance on simulated metabolomics data, the results of which can be found in the Supplementary Information section.

Across the nine datasets under analysis, between 38% and 85% of the simulated-missing metabolites entirely masked from a target dataset were well-predicted with the MIRTH approach (ρ>0, q<0.05 in >90% of trials, FIG. 3d, Table S4). Similar to the within-dataset imputation, performance degraded as a larger proportion of features was simulated as missing (Figure S3b). Properties of the raw data, such as the variance of the feature in the target dataset or the number of samples in other datasets where the feature was measured, partially explained why some features were better predicted than others (Figures S3c,d). Similar to the within-set imputation, MIRTH reliably predicted the ranks of certain metabolites regardless of the target dataset (FIG. 3e). There were 218 reproducibly well-predicted metabolites, consisting of 56 amino acids, 13 carbohydrates, 10 cofactors and vitamins, 94 lipids, 15 nucleotides, 18 peptides, 10 xenobiotics, and 2 uncharacterized metabolites (FIG. 3e, Table S5). Similar to the within-dataset imputation, reproducibly well-predicted metabolites were enriched for lipid subsets and amino acids (FIG. 3e). Tyrosine and palmitate, for instance, were reproducibly well-predicted with a median ρ of 0.892 and 0.845 respectively (FIG. 3f). These results outline a set of metabolites that are likely to be reliably imputed in a new target dataset if one were to be added to our existing aggregate set.

### 2.3 MIRTH Embeddings Separate Tissue of Origin and Metabolite Class

MIRTH involves a matrix factorization that associates both metabolite features and samples with a small number of embedding dimensions. In other contexts, analysis of features and samples in embedding space can be used to interpret the similarity between samples or the covariation of different features. We therefore applied MIRTH jointly to all data available, factorizing the complete aggregate set (X) of all nine datasets. The optimal number of dimensions for the factorization of the aggregate data matrix into embedding matrices W and H was 30 (Figure S4a). Weights were mostly small and right-skewed (Figure S4b). We used UMAP to visualize the sample and feature embedding spaces [20].

In sample embedding space, some clustering occurred by tissue of origin, with samples from the three kidney cancer datasets, RC12, RC18, and RC3, overlapping (FIG. 4a). COAD samples also separated from other tissues of origin. Interestingly, PrCa samples separated into three distinct clusters, raising the possibility of a latent batch effect, i.e. that the PrCa dataset consists of three sub-datasets that are not preprocessed individually by MIRTH. Definition between other tissue types, i.e. between BrCa1 & BrCa2, PaCa, and HCC, was less discernible. Tumor and normal samples from the same dataset also separated in embedding space along UMAP axis 2 (FIG. 4b).

Dimensionality reduction of the feature embedding matrix also revealed separation between certain metabolite classes, in particular of peptides and lipids from the rest of the measured features (FIG. 4c). The outlying peptide features pre-dominantly represented dipeptides (Figure S4c). To determine whether individual embedding vectors were associated with functionally-related groups of metabolites, we performed a Fisher's exact test for enrichment of a given metabolic pathway in each embedding vector after setting a cutoff value above which a feature was considered to be appreciably weighted (here, weight=0.2). This analysis was limited by statistical power, due to the relatively small number of metabolites in each annotated pathway. Nevertheless, the analysis identified enrichment of certain metabolite classes across multiple embedding dimensions, including dipeptides, sphingomyleins, diacylglycerols, and lysolipids (FIG. 4d).

### 2.4 MIRTH Imputes Metabolites Across MS Ionization Modes

Mass spectrometry-based metabolomics can be conducted in positive or negative ionization modes, which allows for quantification of metabolites that are more amenable to acquiring a positive or negative charge [21-23]. We devised an experiment to assess the viability of predicting positive-mode measurements from negative-mode ones (or vice-versa), using a dataset where samples were profiled in both modes [22].

This dataset consisted of 448 features across 638 samples. Of the 241 quantified metabolites, 191 were measured in both positive and negative modes (accounting for 398 features due to redundancies). Of the remaining metabolites, 24 were measured only in positive mode and 16 were measured only in negative mode. We devised a test scenario for MIRTH whereby positive- or negative-mode measurements were completely masked from half the samples. This simulated a scenario where half of the samples were measured in both modes and the remaining samples were measured in just one mode (FIG. 5a). Imputation performance was assessed on metabolites only measured in one mode across 200 trials with a different set of samples chosen for masking each time. All non-overlapping metabolites were well-predicted (ρ>0, q<0.05 in >90% of trials). Overall, negative-mode features were predicted with a higher ρ than positive-mode features (FIG. 5b), perhaps due to the greater reproducibility of the positive-mode measurements on which negative-mode predictions were based [22]. Predictions for glyceraldehyde-3-phosphate, cadaverine, and putrescine—features measured only in positive mode-were notably accurate, with median ρ values of 0.765, 0.788, and 0.777, respectively (FIG. 5c, Table S6). Likewise, aconitate, carbamoyl phosphate, and riboflavin were among the best-predicted negative-mode features, with median ρ values of 0.901, 0.888, and 0.887 respectively (FIG. 5c, Table S6). Once again, these results indicate that MIRTH can impute the ranks of metabolites that were entirely unmeasured by leveraging latent information in metabolomics data—in this case, in one ionization mode.

### 3. Discussion

MIRTH is a novel method to impute the ranks of otherwise unmeasured metabolites in semi-quantitative mass spectrometry metabolomics data by applying a matrix factorization approach tolerant to missing data. MIRTH successfully imputed (ρ>0, p<0.05 in >90% of trials) between 38% and 85% of the missing metabolites in each dataset we tested. Although not all metabolites were well-predicted in all datasets, the existence of a subset of metabolite features that were reproducibly well-imputed across datasets reveals the promise of MIRTH for filling-in missing metabolites in new datasets.

That MIRTH imputes some metabolites poorly may partially be accounted for by the variance of those features across a dataset's samples. Furthermore, the number of datasets the metabolite appeared in and the extent to which the metabolite was left-censored in each batch affected the imputation performance on certain metabolites. These circumstances both create situations in which there is little information on which the model can train for that feature. As can be expected when working with real data, some datasets we tested on had latent structure that strongly shaped metabolite abundance and could, in the future, be included in more sophisticated models based on MIRTH. For example, BrCa1 & BrCa2 samples had variable estrogen receptor (ER) positivity, a metabolically-relevant stratification that MIRTH does not account for; the PrCa dataset consisted of three subgroups of data with apparently strong batch effects.

### 3.1 MIRTH Finds Latent Information in Existing Metabolomics Data

The ability of MIRTH to impute completely unmeasured metabolite features may reduce the cost and complexity of metabolomic profiling. As we have demonstrated, MIRTH can recover rank-normalized metabolite abundances which are of biological or clinical interest without requiring additional tissue or additional proling. This enhances the potential for discovery by enriching publicly-available metabolomics data with additional metabolite features. A key consideration here will be sample size: our success in inferring positive-/negative-mode metabolites (FIG. 5) was in part related to the large number of samples available for training in this dataset.

More generally, the success of MIRTH implies that information on a restricted set of metabolites is sufficient for the imputation of a much larger set of metabolites. We envision the development of an assay which, instead of comprehensively profiling thousands of metabolites in a single sample, simply seeks to measure a small but highly informative subset of metabolites. With accurate measurements of a small panel of predictive metabolites combined with other datasets which measure a wider profile of metabolites, MIRTH or related methods in the future may be able to offer a much wider view of the metabolome at a greatly reduced cost.

### 3.2 MIRTH Embeddings Encode Biological Information

The decomposition of metabolomics data into a product of two low-dimensional matrices empirically captures some aspects of underlying biology. For example, the separation of tumor and normal samples in embedding space suggests that the MIRTH can learn general differences in the metabolome between these types of samples across cancer types. Similarly, since each embedding vector is a parts-based representation of the underlying data, the feature embedding vectors can be considered to represent different “components” of the metabolome, which are then linearly combined according to the sample embeddings to recover the metabolite ranks of a given sample. Furthermore, MIRTH embeddings appear to discern chemical classification of metabolites without incorporating any additional information; for example, the separation of lipids and dipeptides in embedding space hints at high covariance between members of these metabolite classes. Future work could incorporate prior information into the matrix factorization, such as additional information on metabolite classes and structural similarities or on tissue and samples types.

The analysis of the embeddings also provokes questions about the general nature of correlations between metabolite pool sizes. While the existence and characteristics of such correlations are abundantly described in the literature [24{26], neither the mechanistic basis from which they arise nor their generality across biological contexts (e.g. different tissues, or different cancer types) is understood. The general principles which explain how metabolite pools co-vary have been difficult to discern because metabolite pools are subject to complex regulation, both by the metabolic enzymes that produce and consume them, as well as by more distal changes in metabolic flux or cellular physiology. The analysis of the MIRTH embeddings suggests that a relatively small set of linear combinations of metabolite pool sizes is sufficient to describe a large fraction of all the variation in the bulk metabolome. Understanding whether these embeddings are a reflection of a more fundamental, global relationship between metabolite pools is a worthy question for future investigation.

### 3.3 Outlook and Conclusions

The experiments described in this paper suggest that embedded within every metabolomics dataset is latent information about otherwise unmeasured metabolite features. Future work fully harnessing this latent information will likely require overcoming at least two challenges. The first relates to the inherently semiquantitative nature of metabolomics data, in which pool sizes are reported in ion counts that can only be compared across samples within the same feature. MIRTH overcomes this challenge by rank-transforming each metabolite feature within each batch. The cost of this solutions is the loss of information on the magnitude of fluctuations in pool sizes. Future work which instead preserves relative magnitudes while remaining amenable to modeling across batches of data will prove powerful. The second challenge relates to the very likely possibility that some correlations between metabolites will be specific to a particular tissue, disease, or other biological context. Further generalizations of NMF, including those which leverage additional information about the tissue source or disease of interest, prior information on the relationship of metabolites to one another in the metabolic network, or a secondary dataset (e.g gene expression), should be attempted to improve the performance of MIRTH.

### 4. Methods

MIRTH imputes missing metabolites across K metabolomics datasets. Each of I=1 . . . . K datasets Di contains the relative abundance levels of a subset pi of the total metabolites P measured in nᵢ samples. The relative abundance levels are not comparable across metabolites or datasets. MIRTH overcomes this limitation by transforming relative abundance levels to a common scale (normalized ranks) within each batch. MIRTH then applies a nonnegative matrix factorization algorithm to the transformed matrix. By learning latent factors for each metabolite and sample, MIRTH is able to impute missing metabolites both within the same dataset and across datasets.

We have implemented MIRTH in Python v. ≤3.7. A script for MIRTH imputation, as well as a scaled-down demonstration of imputation performance, is available in our Github repository: https://github.com/reznik-lab/MIRTH. Experiments are run on Memorial Sloan Kettering Cancer Center's High-Performance Computing Juno cluster. Figures are generated in R.

We assume that we are given K datasets, each representing one batch of data, i.e. a collection of samples from one metabolomics experiment. Each dataset records measurements of different sets of metabolites with different proportions of metabolite classes represented (Figure S5a). Every entry in the dataset contains a raw ion count for each metabolite detected in a sample. Ion counts below a threshold are not detected by the mass spectrometer. These counts are left-censored; the only information about them is that they are smaller than the smallest reported ion count in that dataset.

In the MIRTH method, the datasets individually undergo normalization and rank transformation accounting for left-censoring as described below (FIG. 1a). Then, the preprocessed single datasets are aggregated into a multiple-dataset matrix, which is then factorized and imputed (FIG. 1b).

### 4.1 Handling Missing Values in Raw Datasets

A targeted metabolomics dataset features two forms of missing data. The first class of missing data corresponds to metabolites which exist in a biological specimen at physiologically-relevant concentrations, but which have not been measured. We refer to these data as “missing metabolites”, emphasizing that their abundance is missing in all samples in a given dataset. The goal of MIRTH is to impute these missing data.

The second corresponds to metabolite measurements that are missing in some samples, but are measured in other samples in the same dataset. These missing values often represent instances where a metabolite's abundance falls below the lowest quantified abundance of that metabolite across all samples. We refer to such instances as “left-censored” measurements. The extent of left-censoring varies by feature and by dataset (Figure S5b). MIRTH has specific procedures which handle left-censored data, as described below.

### 4.2 Normalization

A variety of normalization techniques are used to control for variation in sample loading in metabolomics data [27]. We compared MIRTH's imputation performance with total ion count (TIC) normalization, probabilistic quotient normalization (PQN) and without normalization enabled. MIRTH performs comparably with both normalization methods (Figure S3e). For all analyses described in the text, each dataset was preprocessed with TIC normalization. In TIC normalization, the ion count for every metabolite entry in sample i is normalized by

$${D}_{i}^{N} = \frac{{D}_{i}}{{f}_{{D}_{i}}}$$

where Dᵢ^{N} is the TIC-normalized sample vector, Di is the unnormalized sample vector, and f_{Di} is the TIC normalizer for sample i. The TIC normalizer is computed by summing the ion counts of all j metabolites in the sample,

$${f}_{{D}_{i}} = {\sum }_{j = 1}^{m} {d}_{i , j} + 0.5 \times min ( {D}_{i} ) \times {N}_{{censored}_{{D}_{i}}}$$

where min(Di) is the minimum value in dataset Di and N_{censored} in the number of left-censored entries in the sample. Thus, left-censored values are included in the sum as one-half the minimum value in the dataset.

### 4.3 Rank-Transformation

Since metabolomics only semi-quantitatively measures metabolite pool sizes, only one form of comparison (between two samples' measurements of the same metabolite in the same dataset) is admissible. In contrast, comparisons of the abundance of two metabolites in the same sample or comparisons of the same metabolite across two samples from two different datasets are inadmissible.

We rank the metabolite abundances of all the samples within each dataset. This distributes the sample abundances in each metabolite in the same way, allowing for the comparison of correlations between metabolite abundances in the same dataset and for the transfer of such correlations across datasets (and, therefore, across batches). The samples with the highest ion count for a metabolite in the given dataset are ranked highest. The samples with the lowest ion count are ranked lowest. Left-censored values are tied for last rank.

The rank of the ith uncensored sample in a feature, dᵢ, is found by:

$$rank ( {d}_{4} ) = \frac{1 + {\sum }_{k = 1}^{{N}_{total}} 𝕝 [ {d}_{i} > {d}_{k} ]}{1 + {N}_{total}}$$

where Nₜₒₜₐₗ is the total number of samples in the feature. The rank for the left-censored entries in a feature is set to

$$\frac{0.5 \times ( 1 + {N}_{censored} )}{1 + {N}_{total}}$$

where N_{censored} is the total number of censored samples in the feature. This rank is halfway between the minimum rank of uncensored samples and zero. This maps the features with only uncensored metabolites uniformly from 0 to 1. Following rank-transformation, features in each dataset have the same marginal distribution conditioned on having the same sample size. Rank-transformation results in higher performance compared to simply scaling each feature from 0 to 1 in each dataset when imputing features across datasets (Figure S3f).

### 4.4 Nonnegative Matrix Factorization (NMF)

Nonnegative matrix factorization (NMF) is commonly used to obtain a low-rank approximation of nonnegative, high-dimensional data matrices [28]. NMF decomposes a matrix X∈R^{m×n}, X>0 into W∈R^{m×k} and H∈R^{k×n} such that X≈W H, W, H>0. When the rows of X contain samples, then the columns of factor W describe the relative contributions of each embedding vector to a sample [15] and can reveal clustering among samples [17]. Similarly when the columns of X contain features (metabolites, in this case), the rows of factor matrix H describes the relative contributions of the features to an embedding vector [15, 17].

To prepare the data for NMF, datasets preprocessed with normalization and rank-transformation are aggregated into a single aggregate data matrix X∈Rm×n, with rows corresponding to individual samples and columns corresponding to the complete set of metabolite measured across the batches. The sparseness of X depends on the sparseness and feature overlap of the datasets that comprise it. For example, the matrix X consisting of the nine metabolomics data datasets under consideration has 1727 samples, 1904 metabolite features, and 79.4% missing entries, including both missing metabolites and left-censored entries (FIG. 1c). In MIRTH, we formulate metabolite imputation of the unmeasured metabolites as a nonnegative matrix factorization problem which handles missing values,

$${min}_{W , H} {\sum }_{i = 1}^{m} {\sum }_{j = 1}^{n} {( {x}_{ij} - {ω}_{i}^{T} {h}_{j} )}^{2}$$

$$subject to$$

$$W , H \geq 0$$

where the original data matrix is factored into the product of two low-dimensional matrices W∈R^{m×k} and H∈R^{k×n}, missing xij are omitted in the loss function. The structure of NMF naturally allows the imputation of missing values. Because W and H have fewer entries than X, not all the entries of X are required to perform the decomposition. Provided the loss function is an entry-wise sum of losses, such as the least squares error used here, the matrix can be factorized by dropping the terms corresponding to the missing entries from the loss function [29]. This minimization problem is solved with SciPy's optimize.minimize [30] and the autograd-minimize wrapper [31] with the L-BFGS-B algorithm [32]. Equivalently, a version of scikit-learn NMF that handles missing values can be used for faster runtimes [33]. Solving the optimization problem above produces two matrices W and H, each with no missing entries, whose product \hat{X}=W H also contains no missing entries. Following reconstruction, \hat{X} is rank-transformed again to ensure feature measurements remain mapped uniformly between 0 and 1. These entries are the predicted metabolite ranks imputed by MIRTH.

### 4.5 Cross-Validation

In order to determine the optimal number of embedding dimensions k, we perform v-fold cross-validation; typically, v=10. We evaluate performance over a range of k in [1,80] or [1,60] in the single-dataset or across-dataset imputation cases, respectively.

For each k, we first identify the metabolite features that are available to use to score in dataset-wise cross validation, i.e. the metabolites in each dataset that are also measured in at least one other dataset. These available features are then equally partitioned into v folds (i.e., sets of metabolite features in each dataset on which we will test model performance with different sets of cross-validation parameters). Within each dataset, metabolites are randomly assigned to folds in order to reduce the amount of overlap between folds in different datasets (Figure S5c). Once the folds are denied, MIRTH loops through them, treating one at a time as unmeasured (masking all the fold's features in the datasets where they appear), and factorize the resulting matrix. We then take the product WH to recover \hat{X}, the imputed matrix with no missing data. Next, we compute the mean absolute error (MAE) between the true ranks of the metabolites in the fold (the metabolites we simulated as unmeasured) and the imputed ranks of those metabolites; the MAE is treated as the performance score for that fold. The scores for each fold are averaged, which yields a score for the particular value of k. This process is repeated for all k, and the value which results in the lowest MAE is chosen as the optimal number of embedding dimensions for the factorization (Figure S5d).

### 4.6 Evaluating MIRTH's Performance

To evaluate MIRTH's predictions of metabolite ranks, we mask a subset of metabolite measurements to simulate them as missing. After imputing the masked data with MIRTH, we compare the imputed metabolite ranks to the true ranks of the features that were simulated as missing. The chosen performance metric is Spearman's rank correlation coefficient (ρ), computed between actual and predicted metabolite ranks. Correlation coefficients are computed separately for each metabolite that was simulated as missing. The resulting ρ values are then summarized, either across all the metabolites simulated as missing in one experiment (to assess the overall prediction quality of a single imputation) or for the same metabolite across several repeated experiments (to assess metabolite-specific prediction quality). The ρ values are Fisher z-transformed before summarization; the summarized z-scores are then inverse-z-transformed to yield summarized ρ values. P-values are adjusted for multiple testing with Benjamini-Hochberg (BH) correction [34]. Details on how metabolite ranks are simulated as missing are included in their respective Results sections. Metabolites which are predicted with significant positive correlations with true ranks in more 90% of trials are deemed well-predicted. Metabolites are deemed reproducibly well-predicted if they are measured in at least four datasets and are well-predicted in at least three-quarters of the datasets in which they are measured.

### 5. MIRTH Experiments with Simulated Data

### 5.1 Generating Simulated Data

Generating simulated data can accelerate the development of machine learning methods when acquiring real training data is costly [35]. The nine benchmark datasets in this study have variable size and sparsity. Evaluating MIRTH's performance on realistic, synthetic datasets can help parse the effect of size and sparseness on performance under controlled conditions. As such, we simulated metabolomics datasets for benchmarking.

Our analysis confirmed that MIRTH's multidimensional embedding space clusters related samples and metabolite features (FIG. 4a-c). Consequently, a simulated dataset {s1, . . . , sN} would consist of n observations of a random m-dimensional Euclidean variable, such that the data belongs to J clusters.

We use a generative model to simulate metabolomics datasets, S∈R^{m×n} where ^{m} is the number of samples and ⁿ is the number of metabolites. The dataset is generated as the product of simulated embeddings matrices, W sim∈R^{m×k} and Hₛᵢₘ∈R^{k×n} where ^{k} is the number of embedding dimensions. To generate the embeddings matrices, a k-dimensional multivariate distribution of J cluster centroids is generated. This distribution is sampled to set the center of each embedding vector. The dataset is simulated as follows,

$$To generate {W}_{sim} :$$

$$\text{?} Gamma ( k = 100 , θ = 1 ) \qquad ( 2 )$$

$${d}_{n} \text{?} Cat ( α )$$

$$ω \text{?} ∼ Gamma ( k = 100 , θ = 1 ) + c \text{?}$$

$$To generate {H}_{sim} :$$

$$\text{?} Gamma ( k = 100 , θ = \text{.1} ) \qquad ( 3 )$$

$${d}_{m} \text{?} Cat ( α )$$

$$h \text{?} ∼ Gamma ( k = \text{.1} , θ = 1000 ) + c \text{?}$$

$$To generate S :$$

$$S = {W}_{sim} \times {H}_{sim} \qquad ( 4 )$$

$$where {α}_{j} = 1 , j = 1 … {j}_{clusters} .$$

$$\text{?} \text{indicates text missing or illegible when filed}$$

The matrix product of these two embedding matrices constructs a simulated dataset. A set of feature names to sample from is defined. The columns of the dataset are randomly assigned a subset of these feature names. The rows of the dataset are given labels according to dataset and sample number. Once labeled, the dataset can then be combined into an aggregate data matrix (X) or used in a single-set imputation. We generated data with j_{clusters}=5 and k=10. The shape, k, and scale (Ø) of the sampled distributions for both the centroids and embedding matrix weights were chosen to create data similar to the 9 real datasets used for benchmarking.

### 5.2 Experiment 1: Size

In this experiment, nine simulated datasets of identical size to the real datasets were generated. To isolate the effect of dataset size on within-dataset imputation performance. First, we applied MIRTH to one dataset at a time, masking 10% of the features in half the samples (FIG. 2a). These masked entries in each dataset were imputed. Performance was better for larger datasets (Figure S6a), indicating that larger sample size improves within-set imputation performance. In general, the simulated data was imputed with much higher median ρ than real data.

To isolate the effect of dataset size on cross-set imputation performance, we applied MIRTH to the aggregate dataset (9 merged simulated datasets), holding out entire features in one target dataset at a time (FIG. 3c). The cross-dataset imputation performance on the simulated data was roughly constant while the performance on the real data varied depending on the target dataset (Figure S6c). This simulation indicated that factors other than size contribute to performance of the across-set MIRTH imputation.

### 5.3 Experiment 2: Missingness

To test the effect of randomly-missing data on MIRTH's imputation performance, identically-sized datasets were generated. Entries were randomly masked, with the proportion of masked entries ranging from 0% to 90% with a 10% step size. We evaluated single-set MIRTH imputation as usual (FIG. 2a). As the number of missing entries increases, the median performance (ρ) approached 0 (Figure S6b). The low performance stabilized around 50% missing entries.

We also constructed three datasets of identical size whose entries were randomly masked at different proportions (10%, 50%, 90%). We evaluated MIRTH's across-set imputation performance as usual (FIG. 2c). As the number of randomly-missing entries increased, the median performance (ρ) decreased, approaching 0 (Figure S6d).

### 5.4 Experiment 3: Censoring

To simulate left-censoring, the ten lowest-valued entries in each feature in a dataset are masked. The imputed ranks of the simulated-censored measurements are compared to the true ranks of the same samples. Alternatively, the imputed ranks of the unmasked ten-lowest values can be compared to their true ranks.

We evaluated censoring imputation on real data (RC12). There are already many censored measurements in the dataset. Imputed ranks are consistently lower when their corresponding measurements are simulated as censored than when they are actually included in the training data (Figure S6e). Since some features have only one or a few measurements, a cluster of true ranks appears near 0.5.

This evaluation scheme was also run on a simulated dataset with 30% of entries randomly missing. Similarly, imputed ranks are lower when the data point is simulated censored than when the data point is actually included in the training data (Figure S6f). These findings confirm that MIRTH preserves the low ranks of censored data, which ultimately correspond to low metabolite abundance.

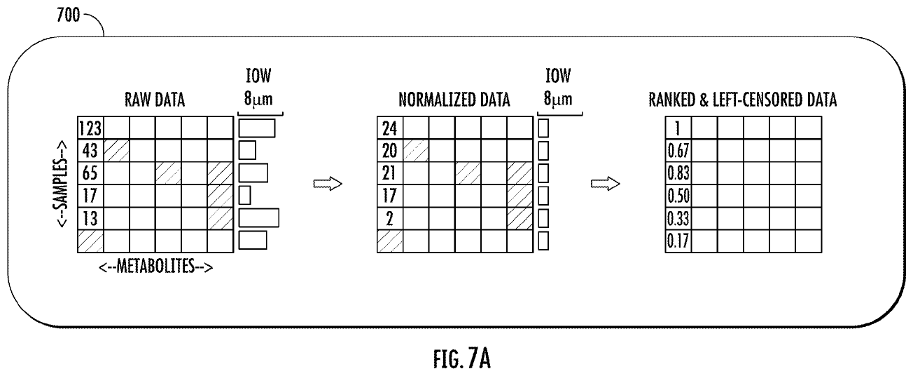

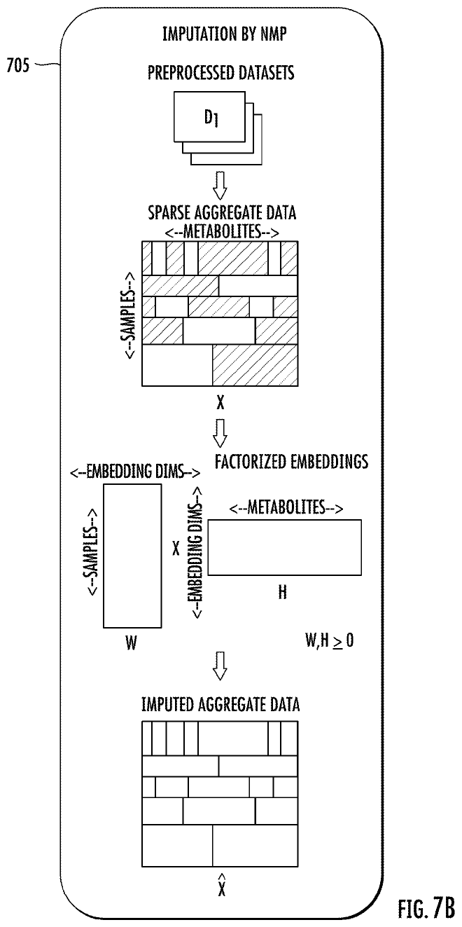

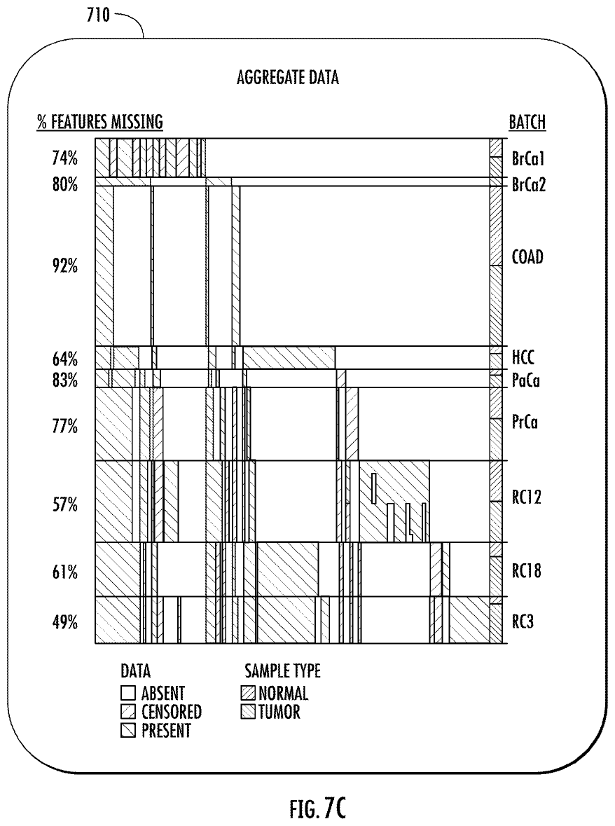

Referring now to FIGS. 7A, 7B, and 7C, among others, a flow diagram for imputing metabolite information is shown. As shown in FIG. 7, individual datasets can be normalized and rank-transformed. The normalized and rank-transformed datasets can account for left-censoring. Preprocessed datasets (Dᵢ) are combined into a sparse aggregate data matrix (X), which is then factorized into embedding matrices W and H. The product WH yields an imputed data matrix (X\hat{ }). Aggregate data from 9 pan-cancer metabolomics datasets with tumor and normal samples reveals poor across-dataset metabolite feature overlap and high degree of missingness, for example.

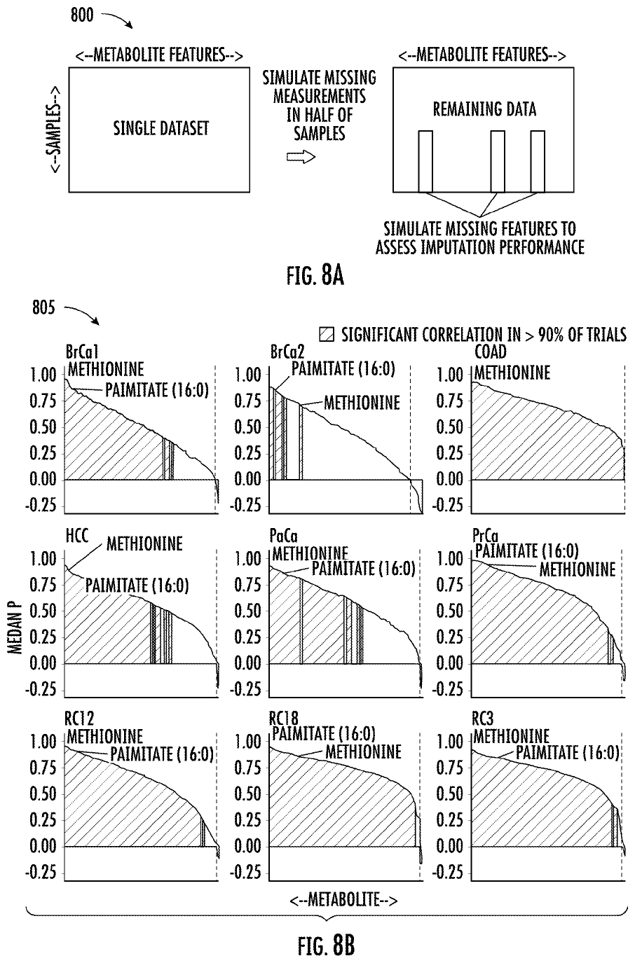

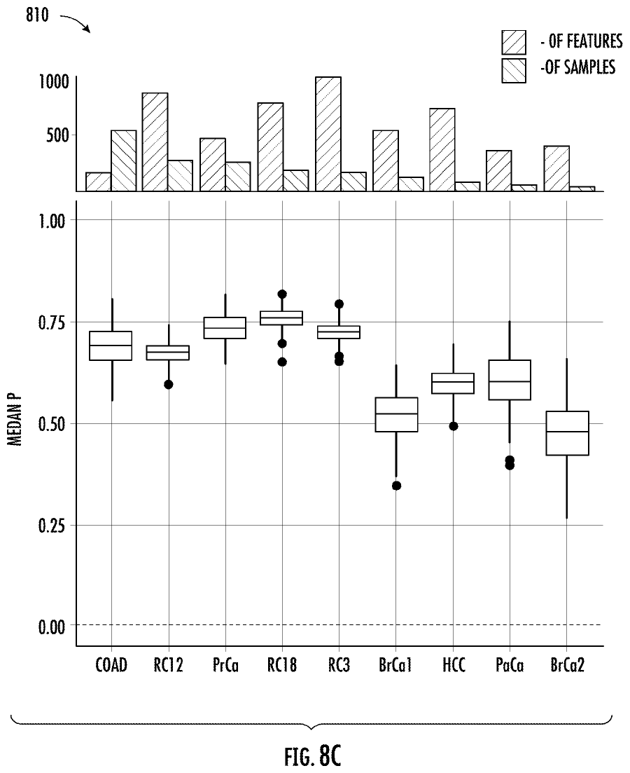

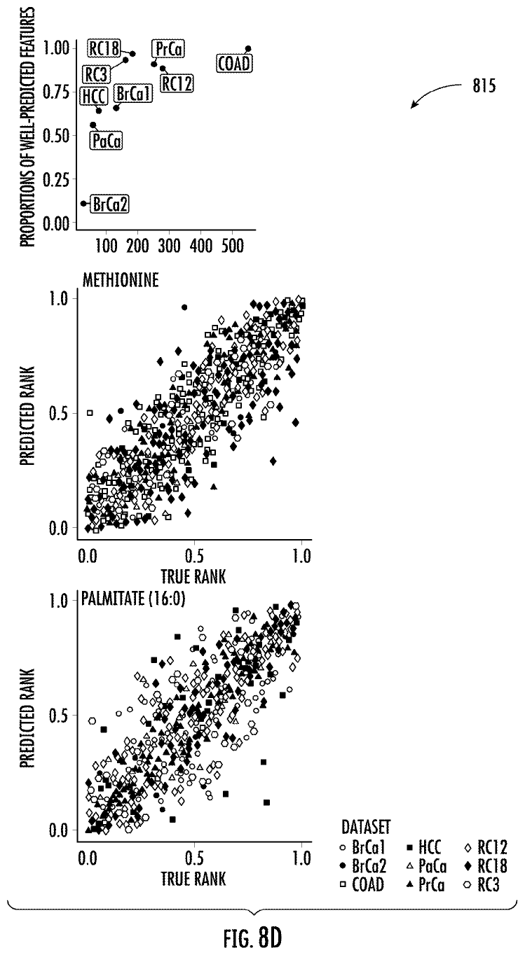

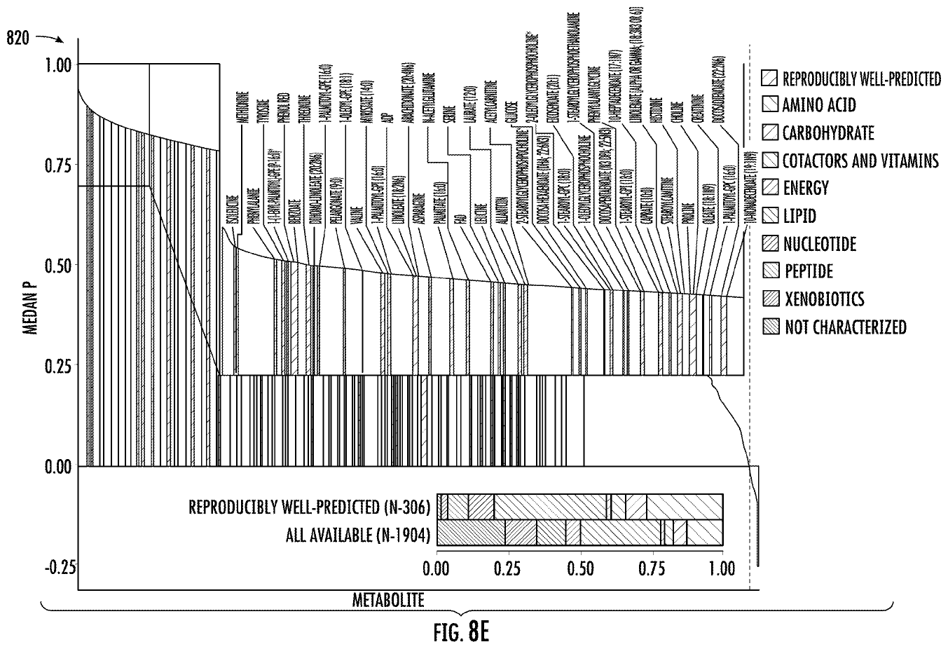

Referring now to FIGS. 8-11, among others, sample datasets and related graphics generated by a metabolite computing system, such as the computing system 205. As shown in FIGS. 8A, 8B, 8C, 8D, and 8E, the computing system 205 can achieve high accuracy imputing within datasets, such as metabolite datasets. In one example, samples for a subset of features were masked in half of all samples in a dataset before imputation to create data on which to assess imputation performance. Imputation performance by dataset is reported by median ρ values across all simulated-missing features in each iteration. Imputation performance by metabolite, reported as the median ρ value for each metabolite across all trials, is plotted for each batch is shown. As dataset size (number of samples) increases along the x-axis, the proportion of well-predicted metabolites in a batch increased as well. Imputation performance for each metabolite summarized across datasets (median ρ values across datasets are plotted) is shown. A subset of consistently well-imputed metabolites are provided. Reproducibly well-predicted metabolites are indicated in blue. The true ranks versus the predicted ranks of example metabolites, methionine and palmitate (16:0), when imputed in each single dataset

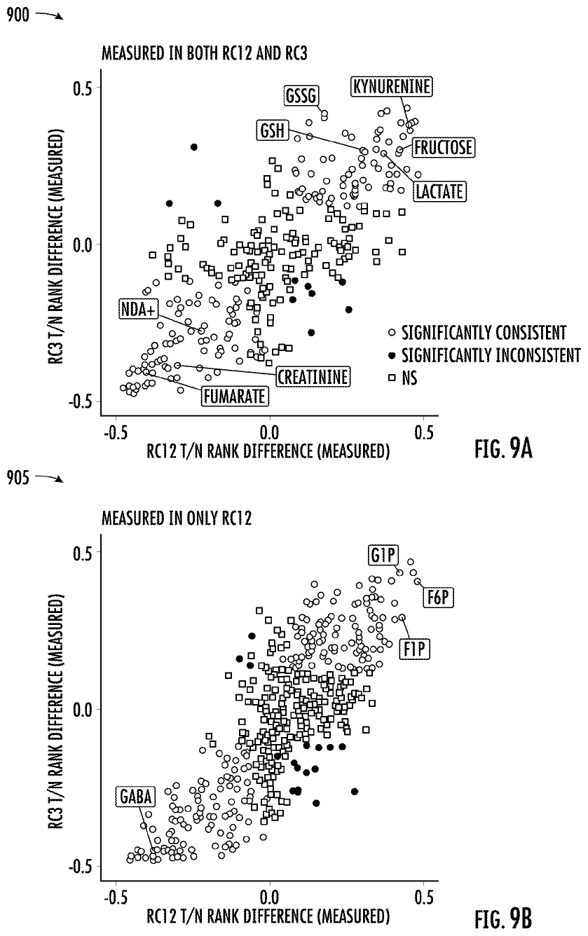

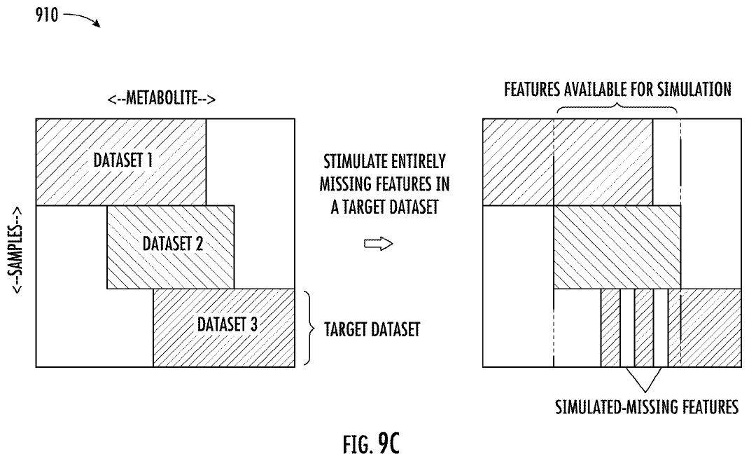

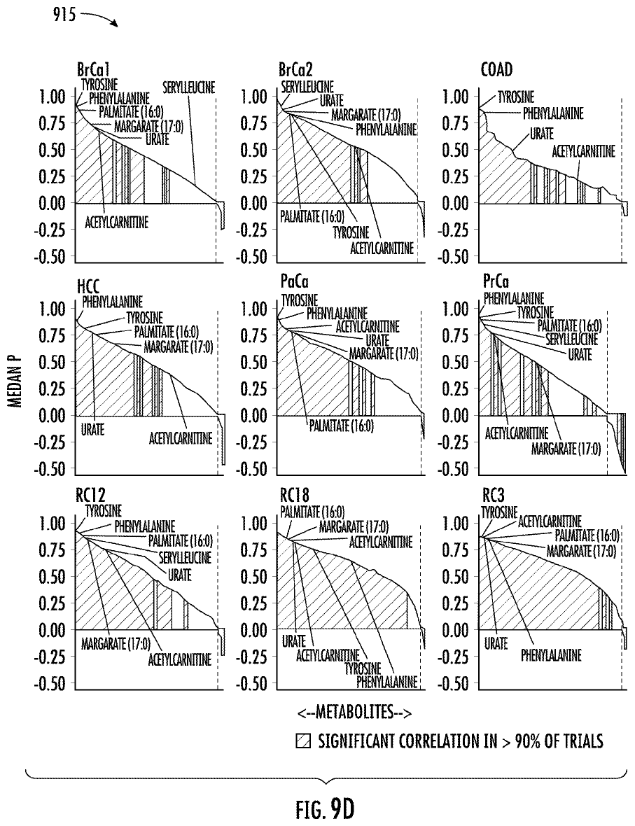

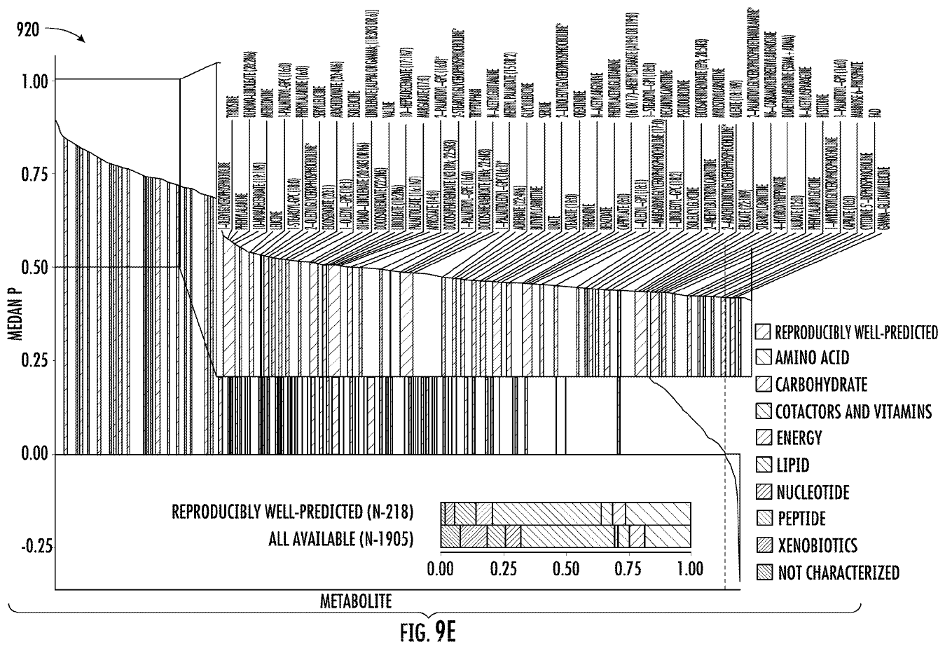

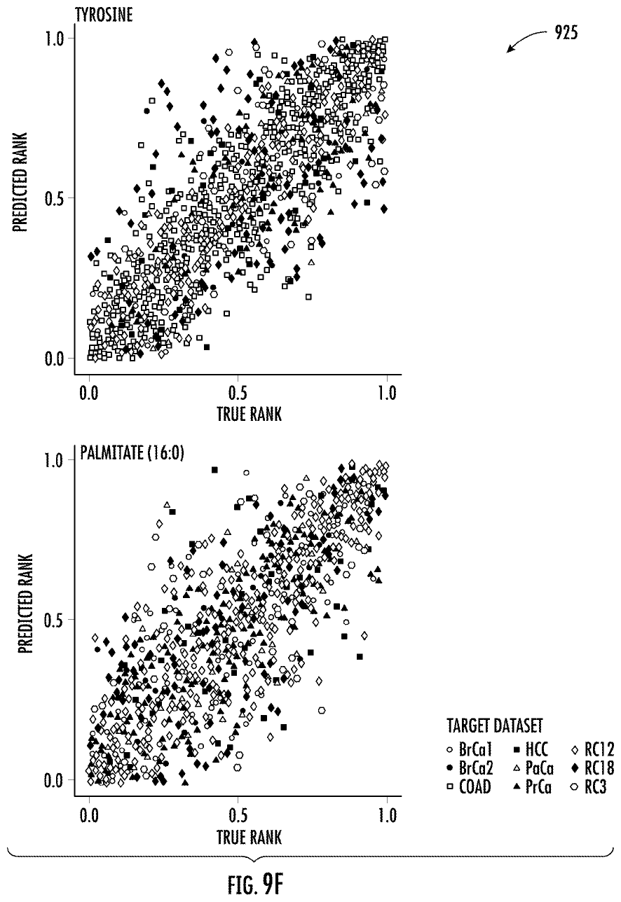

As shown in FIGS. 9A, 9B, 9C, 9D, 9E, and 9F, the computing system 205 can achieve high accuracy in cross-dataset imputation and preserves biological characteristics in the data. The same metabolites distinguish tumor and normal samples in RC12 and RC3, for example, and tumor-distinguishing metabolite patterns can be recovered by imputation of RC3 and RC12. A schematic of experiment to assess the imputation of features that were entirely unmeasured in a dataset is shown. Typical by-metabolite ρ values when those metabolite features are entirely masked from a target dataset. e. By-metabolite performance summarized across target datasets, showing that many of the same metabolites are well-predicted in many target datasets. A relationship between actual and predicted within-dataset metabolite ranks for two well-predicted metabolites is provided.

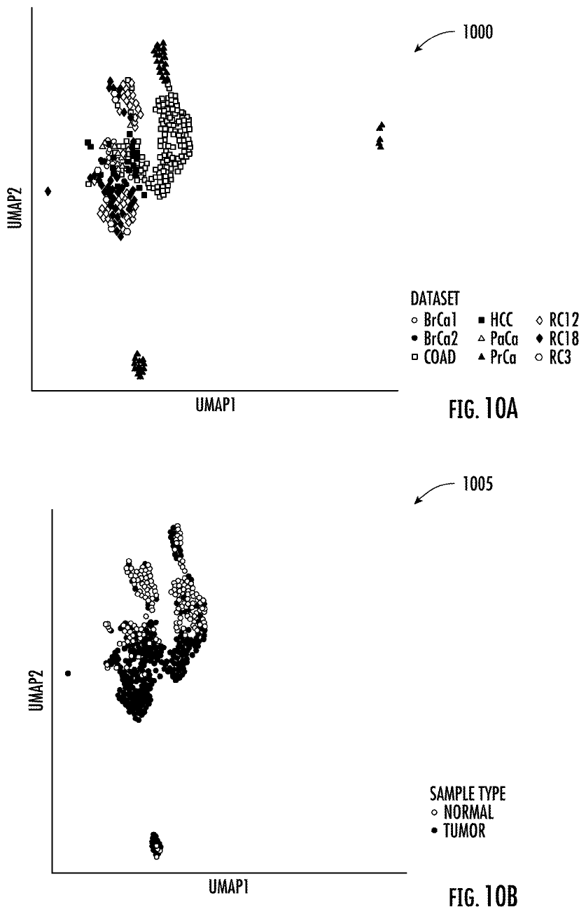

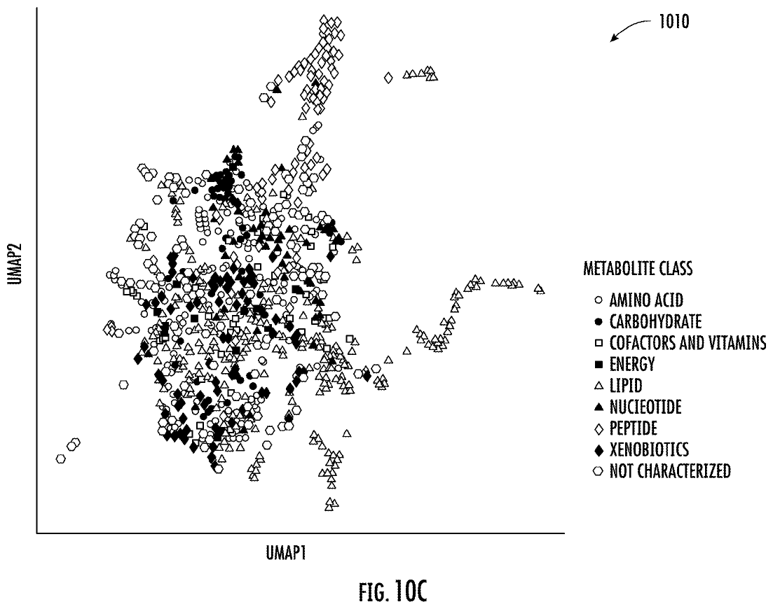

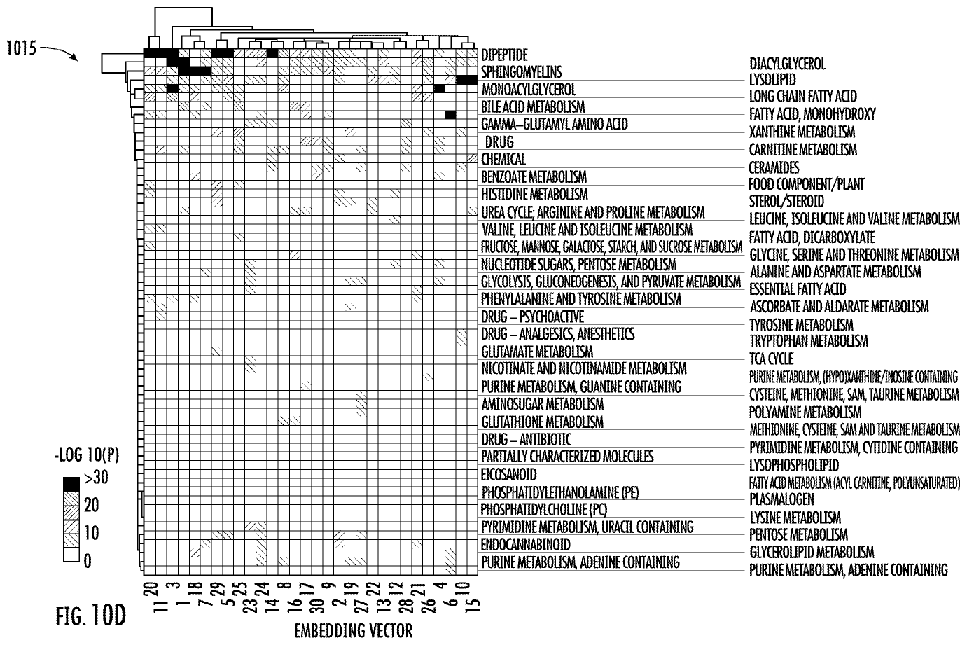

As shown in FIGS. 10A, 10B, 10C, and 10D, embedding matrices reveal separation of features and samples and enrichment in certain metabolic pathways. UMAP plots of sample embedding matrix W reveal some separation between batches and cancer types, as well as separation between tumor and normal samples, as shown. Feature embeddings separate peptides and lipids from other metabolites, for example. Certain pathways are enriched in certain embedding dimensions, though analysis is limited by statistical power.

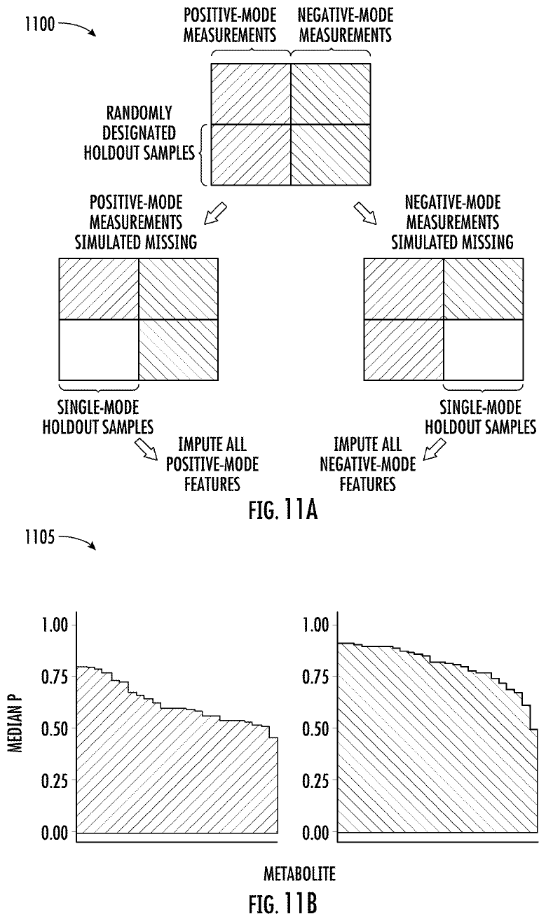

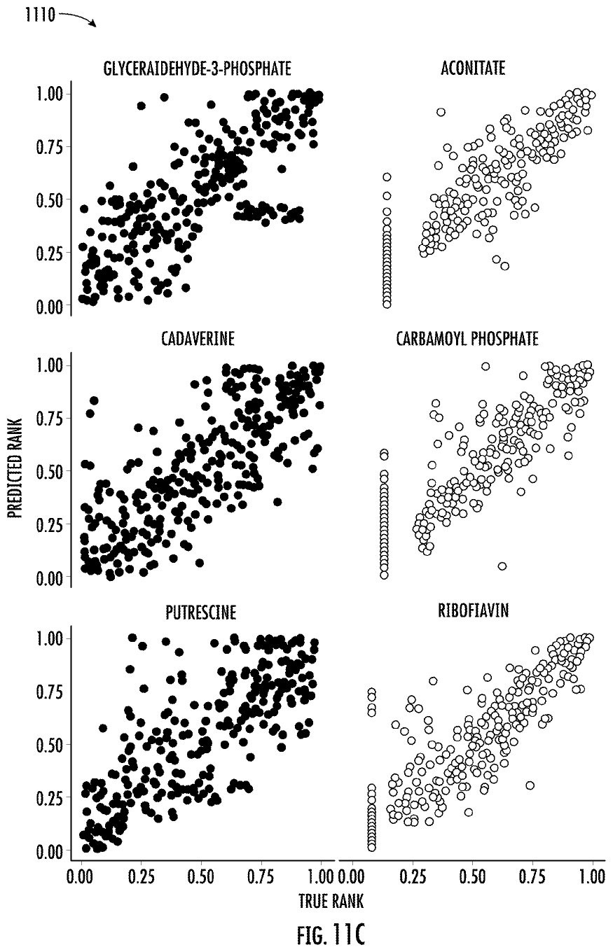

Referring to FIGS. 11A, 11B, and 11C, among others, the computing system 205 can accurately impute across features measured in different mass spectrometer ionization modes. Simulating a subset of samples as only measured in a single ionization mode, then imputing to assess performance. Summarized imputation performance by ionization mode. Only metabolites measured in a single ionization mode are shown. Examples of metabolites that are well-predicted across ionization modes.

### C. Computing and Network Environment

Various operations described herein can be implemented on computer systems. FIG. 6 shows a simplified block diagram of a representative server system 600, client computer system 614, and network 626 usable to implement certain embodiments of the present disclosure. In various embodiments, server system 600 or similar systems can implement services or servers described herein or portions thereof. Client computer system 614 or similar systems can implement clients described herein. The system 200 described herein can be similar to the server system 600. Server system 600 can have a modular design that incorporates a number of modules 602 (e.g., blades in a blade server embodiment); while two modules 602 are shown, any number can be provided. Each module 602 can include processing unit(s) 604 and local storage 606.

Processing unit(s) 604 can include a single processor, which can have one or more cores, or multiple processors. In some embodiments, processing unit(s) 604 can include a general-purpose primary processor as well as one or more special-purpose co-processors such as graphics processors, digital signal processors, or the like. In some embodiments, some or all processing units 604 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 604 can execute instructions stored in local storage 606. Any type of processors in any combination can be included in processing unit(s) 604.

Local storage 606 can include volatile storage media (e.g., DRAM, SRAM, SDRAM, or the like) and/or non-volatile storage media (e.g., magnetic or optical disk, flash memory, or the like). Storage media incorporated in local storage 606 can be fixed, removable or upgradeable as desired. Local storage 606 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 604 need at runtime. The ROM can store static data and instructions that are needed by processing unit(s) 604. The permanent storage device can be a non-volatile read-and-write memory device that can store instructions and data even when module 602 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 606 can store one or more software programs to be executed by processing unit(s) 604, such as an operating system and/or programs implementing various server functions such as functions of the system 200 of FIG. 2 or any other system described herein, or any other server(s) associated with system 200 or any other system described herein.

“Software” refers generally to sequences of instructions that, when executed by processing unit(s) 604 cause server system 600 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 604. Software can be implemented as a single program or a collection of separate programs or program modules that interact as desired. From local storage 606 (or non-local storage described below), processing unit(s) 604 can retrieve program instructions to execute and data to process in order to execute various operations described above.

In some server systems 600, multiple modules 602 can be interconnected via a bus or other interconnect 608, forming a local area network that supports communication between modules 602 and other components of server system 600. Interconnect 608 can be implemented using various technologies including server racks, hubs, routers, etc.

A wide area network (WAN) interface 610 can provide data communication capability between the local area network (interconnect 608) and the network 626, such as the Internet. Technologies can be used, including wired (e.g., Ethernet, IEEE 1302.3 standards) and/or wireless technologies (e.g., Wi-Fi, IEEE 1302.11 standards).

In some embodiments, local storage 606 is intended to provide working memory for processing unit(s) 604, providing fast access to programs and/or data to be processed while reducing traffic on interconnect 608. Storage for larger quantities of data can be provided on the local area network by one or more mass storage subsystems 612 that can be connected to interconnect 608. Mass storage subsystem 612 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 612. In some embodiments, additional data storage resources may be accessible via WAN interface 610 (potentially with increased latency).

Server system 600 can operate in response to requests received via WAN interface 610. For example, one of modules 602 can implement a supervisory function and assign discrete tasks to other modules 602 in response to received requests. Work allocation techniques can be used. As requests are processed, results can be returned to the requester via WAN interface 610. Such operation can generally be automated. Further, in some embodiments, WAN interface 610 can connect multiple server systems 600 to each other, providing scalable systems capable of managing high volumes of activity. Other techniques for managing server systems and server farms (collections of server systems that cooperate) can be used, including dynamic resource allocation and reallocation.

Server system 600 can interact with various user-owned or user-operated devices via a wide-area network such as the Internet. An example of a user-operated device is shown in FIG. 6 as client computing system 614. Client computing system 614 can be implemented, for example, as a consumer device such as a smartphone, other mobile phone, tablet computer, wearable computing device (e.g., smart watch, eyeglasses), desktop computer, laptop computer, and so on.

For example, client computing system 614 can communicate via WAN interface 610. Client computing system 614 can include computer components such as processing unit(s) 616, storage device 618, network interface 620, user input device 622, and user output device 624. Client computing system 614 can be a computing device implemented in a variety of form factors, such as a desktop computer, laptop computer, tablet computer, smartphone, other mobile computing device, wearable computing device, or the like.

Processor 616 and storage device 618 can be similar to processing unit(s) 604 and local storage 606 described above. Suitable devices can be selected based on the demands to be placed on client computing system 614; for example, client computing system 614 can be implemented as a “thin” client with limited processing capability or as a high-powered computing device. Client computing system 614 can be provisioned with program code executable by processing unit(s) 616 to enable various interactions with server system 600.

Network interface 620 can provide a connection to the network 626, such as a wide area network (e.g., the Internet) to which WAN interface 610 of server system 600 is also connected. In various embodiments, network interface 620 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 622 can include any device (or devices) via which a user can provide signals to client computing system 614; client computing system 614 can interpret the signals as indicative of particular user requests or information. In various embodiments, user input device 622 can include any or all of a keyboard, touch pad, touch screen, mouse or other pointing device, scroll wheel, click wheel, dial, button, switch, keypad, microphone, and so on.

User output device 624 can include any device via which client computing system 614 can provide information to a user. For example, user output device 624 can include a display to display images generated by or delivered to client computing system 614. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that functions as both input and output device. In some embodiments, other user output devices 624 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 604 and 616 can provide various functionality for server system 600 and client computing system 614, including any of the functionality described herein as being performed by a server or client, or other functionality.

It will be appreciated that server system 600 and client computing system 614 are illustrative and that variations and modifications are possible. Computer systems used in connection with embodiments of the present disclosure can have other capabilities not specifically described here. Further, while server system 600 and client computing system 614 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method, comprising:
receiving, by a computing system over a network from a database, a first dataset and a second dataset, the first dataset comprising data associated with a first set of metabolites, the second dataset comprising data associated with a second set of metabolites;
normalizing, by the computing system, the first dataset and the second dataset via a total ion count (TIC) normalization;
transforming, by the computing system, the normalized first dataset and second dataset, the transformation ranking at least one left-censored entry of the first dataset or the second dataset;
aggregating, by the computing system, the normalized first dataset and the normalized second dataset to generate a first metabolite matrix, the first metabolite matrix missing a first relative abundance value;
decomposing, by the computing system, the first metabolite matrix into a second metabolite matrix and a third metabolite matrix to factorize the first metabolite matrix; and
generating, by the computing system, a fourth metabolite matrix, wherein the fourth metabolite matrix is a product of the second metabolite matrix and the third metabolite matrix,
wherein the fourth metabolite matrix including an imputed first relative abundance value.

2. The method of claim 1, further comprising:
transforming, by the computing system, the fourth metabolite matrix to uniformly map metabolite features of the fourth metabolite matrix between 0 and 1.

3. The method of claim 1, wherein the first dataset is received from a first remote database and the second dataset is received from a second remote database.

4. The method of claim 1, wherein the missing first relative abundance value comprises a relative abundance value of a metabolite that was not measured in the first dataset or the second dataset.

5. The method of claim 1, further comprising:
applying, by the computing system, a loss function to identify a factorization value, the factorization value dictating a dimension of at least one of the second matrix or the third matrix.

6. The method of claim 5, wherein the loss function is a least squares error loss function, a hinge loss function, or a log loss function.

7. The method of claim 1, further comprising:
identifying, by the computing system, a third dataset likely to improve an accuracy of the imputed relative abundance value when normalized, transformed, and aggregated with the first dataset and the second dataset to generate an updated first metabolite matrix.

8. A computing system, comprising:
one or more processors and one or more memory, the memory storing instructions that, when executed by the one or more processors, cause the one or more processors to:
receive, via a network from a remote database, a first dataset and a second dataset, the first dataset comprising data associated with a first set of metabolites, the second dataset comprising data associated with a second set of metabolites;
normalize the first dataset and the second dataset via a total ion count (TIC) normalization;
transform the normalized first dataset and second dataset, the transformation ranking at least one left-censored entry of the first dataset or the second dataset;
aggregate the normalized first dataset and the normalized second dataset to generate a first metabolite matrix, the first metabolite matrix missing a first relative abundance value;
decompose the first metabolite matrix into a second metabolite matrix and a third metabolite matrix to factorize the first metabolite matrix; and
generate a fourth metabolite matrix, wherein the fourth metabolite matrix is a product of the second metabolite matrix and the third metabolite matrix,
wherein the fourth metabolite matrix including an imputed first relative abundance value.

9. The computing system of claim 8, the instructions further cause the one or more processors to:
transform the fourth metabolite matrix to uniformly map metabolite features of the fourth metabolite matrix between 0 and 1.

10. The computing system of claim 8, wherein the first dataset is received from a first remote database and the second dataset is received from a second remote database.

11. The computing system of claim 8, wherein the missing first relative abundance value comprises a relative abundance value of a metabolite that was not measured in either the first dataset or the second dataset.

12. The computing system of claim 8, the instructions further cause the one or more processors to:
apply, a loss function to identify a factorization value, the factorization value dictating a dimension of at least one of the second matrix or the third matrix.

13. The computing system of claim 12, wherein the loss function is a least squares error loss function, a hinge loss function, a log loss function.

14. The computing system of claim 8, the instructions further cause the one or more processors to:
identify a third dataset likely to improve an accuracy of the imputed relative abundance value when normalized, transformed, and aggregated with the first dataset and the second dataset to generate an updated first metabolite matrix.

15. A non-transitory computer-readable medium with computer-executable instructions embodied thereon that, when executed by at least one processor of a computing system, cause operations comprising:
receiving over a network from a database, a first dataset and a second dataset, the first dataset comprising data associated with a first set of metabolites, the second dataset comprising data associated with a second set of metabolites;
normalizing the first dataset and the second dataset via a total ion count (TIC) normalization;
transforming the normalized first dataset and second dataset, the transformation ranking at least one left-censored entry of the first dataset or the second dataset;
aggregating the normalized first dataset and the normalized second dataset to generate a first metabolite matrix, the first metabolite matrix missing a first relative abundance value;
decomposing the first metabolite matrix into a second metabolite matrix and a third metabolite matrix to factorize the first metabolite matrix; and
generating a fourth metabolite matrix, wherein the fourth metabolite matrix is a product of the second metabolite matrix and the third metabolite matrix,
wherein the fourth metabolite matrix including an imputed first relative abundance value.

16. The non-transitory computer-readable medium of claim 15, wherein the instructions, when executed by the at least one processor, further cause operations comprising:
transforming the fourth metabolite matrix to uniformly map metabolite features of the fourth metabolite matrix between 0 and 1.

17. The non-transitory computer-readable medium of claim 15, wherein the first dataset is received from a first remote database and the second dataset is received from a second remote database.

18. The non-transitory computer-readable medium of claim 15, wherein the missing first relative abundance value comprises a relative abundance value of a metabolite that was not measured in the first dataset or the second dataset.

19. The non-transitory computer-readable medium of claim 15, wherein the instructions, when executed by the at least one processor, further cause operations comprising:
applying a loss function to identify a factorization value, the factorization value dictating a dimension of at least one of the second matrix or the third matrix.

20. The non-transitory computer-readable medium of claim 15, wherein the instructions, when executed by the at least one processor, further cause operations comprising:
identifying a third dataset likely to improve an accuracy of the imputed relative abundance value when normalized, transformed, and aggregated with the first dataset and the second dataset to generate an updated first metabolite matrix.
